(19) United States
(12) Patent Application Publication
(10) Pub. No.: US 2026/0283094 A1
(43) Pub. Date: Sep. 24, 2026

(54) PENTAS PARENT LINES

(71) Applicant: Ernst Benary Samenzucht GmbH, Hann. Münden (DE)

(72) Inventor: David CZARNECKI, II, Gimte (DE)

(73) Assignee: Ernst Benary Samenzucht GmbH, Hann. Münden (DE)

(21) Appl. No.: 19/573,988

(22) Filed: Mar. 20, 2026

Related U.S. Application Data
(60) Provisional application No. 63/775,505, filed on Mar. 21, 2025.

(51) Int. Cl. A01H 6/76; A01H 5/02

(52) U.S. Cl. CPC A01H 6/76; A01H 5/02

(57) ABSTRACT

New and distinctive Pentas parent lines are disclosed. The Pentas “Graffiti® Falls” parent lines have a gravitropic growth habit. The Pentas “Northern Lights®” parent lines have distinctive and bright flower colors. Exemplary parent line varieties are provided for both parent line groups. The present disclosure further relates to methods of using Pentas “Graffiti® Falls” parent lines and Pentas “Northern Lights®” parent lines for producing Pentas hybrids.

### CROSS-REFERENCE TO RELATED APPLICATIONS

This application claims the benefit of U.S. Provisional Application No. 63/775,505, filed Mar. 21, 2025, which is herein incorporated by reference in its entirety.

### FIELD

The present disclosure relates to the field of plant breeding. In particular, the present disclosure relates to new and distinctive Pentas “Graffiti® Falls” parent lines and Pentas “Northern Lights®” parent lines.

### BACKGROUND

Members of the genus Pentas are herbs or shrubs of the Rubiaceae family and are native to Africa and Madagascar. The species Pentas lanceolata (Forssk.) Deflers, also known as star-flower, Egyptian star-flower and Pentas, is commonly used as a cut flower and as a potted or bedding plant. Pentas can be cultivated in greenhouses on a year-round basis and can also serve as bedding plants in the southern part of the United States. Pentas plants generally have dark green, lance-shaped, somewhat furry and deeply veined leaves providing a backdrop for prolific clusters of five-petaled flowers. Pentas lanceolata flowers may be white, pink, violet, or shades of red, and may be two-toned. All are extremely attractive to butterflies, and the red and dark pink varieties have been proven to attract hummingbirds.

While Pentas lanceolata generally has a wide range of flower colors, the horticulturally optimized Pentas clone 99/280-6 (U.S. Pat. No. 7,880,073) only has flowers that are white in color with a slight blush to pink and a prostrate growth habit (the habit is wider than tall, branches are slightly arching). This means that this horticultural breeding line can only be used to produce a limited range of colors in its hybrid offspring. As the attractive appearance of hybrid offspring is a primary factor in their marketability, the limited flower color range has meant that the use of Pentas clone 99/280-6 has also been limited.

Pentas can have different growth habits such as a mound shape, which grows up and out at the same time. However, the mound-shaped Pentas on the market come with some drawbacks, including a lower vigor that leads to lower performance later in the season. Pentas are frequently paired with other ornamental and fruiting plants in various containers to suit many purposes. Pentas with stronger stems would be easier to ship, improving transit in the supply chain. In addition, increased vigor of the stem would make the plants more resilient for outdoor planting with exposure to different weather conditions. Moreover, the commercially available Pentas varieties do not include a variety that is suitable for a hanging basket.

Pentas plants are popular and valuable ornamental plants. Thus, there is a continued need to develop Pentas lines with horticulturally valuable characteristics such as unique flower colors and unique plant growth habits. In particular, there exists a need for Pentas varieties able to capture a new market segment offered by growth in a hanging basket. Growth in a hanging basket is particularly desirable for ornamental plants, because it opens up opportunities for pairing with different ornamental and fruiting plants than standard container or bed plantings, new locations for ornamental plant use (e.g., main street sidewalks), a broader variety of landscaping possibilities (e.g., where plants spill over walls or are planted in elevated beds).

### BRIEF DESCRIPTION OF THE DRAWINGS

The patent or application file contains at least one drawing executed in color. Copies of this patent or patent application publication with color drawings will be provided by the office upon request and payment of the necessary fee.

FIGS. 1A-1D show exemplary images of developmental stages of “Graffiti® Falls” parent line plants. FIG. 1A shows an exemplary image of a 12-week-old “Graffiti® Falls” parent line plant beginning to display gravitropic growth. FIG. 1B shows a second exemplary image of 19-week-old “Graffiti® Falls” parent line plant beginning to display gravitropic growth. FIG. 1C shows another exemplary image of a 19-week-old “Graffiti® Falls” parent line plant stripped of leaves with more developed and pronounced gravitropic growth. FIG. 1D shows an exemplary image of a more mature, approximately 62-week-old, “Graffiti® Falls” parent line, ‘PN25-1-2247’, plant with more developed and pronounced gravitropic growth.

FIG. 2 shows 12-week-old commercial “Graffiti® Falls” (white) parent line plants that do not yet display the gravitropic growth.

FIG. 3 shows an exemplary image of a mature, approximately 22 to 24 week-old “Graffiti® Falls” (Rose) parent line plant demonstrating the trailing growth habit suitable for a hanging basket.

FIGS. 4A-4C show exemplary images of young “Northern Lights®” (Lavendar) parent line plants. FIG. 4A shows an exemplary top view of an approximately 12-week-old “Northern Lights®” (Lavendar) parent line plant. FIG. 4B shows an exemplary side view of an approximately 12-week-old “Northern Lights®” (Lavendar) parent line plant. FIG. 4C shows an exemplary view of multiple 12-week-old “Northern Lights®” (Lavendar) parent line plants.

FIG. 5 shows an exemplary image of a flowering “Northern Lights®” (Lavendar) parent line plant that is approximately 22 to 24 weeks old.

FIGS. 6A-6C show exemplary images of young “Beehive® White” Pentas plants, which are a comparison variety to the “Graffiti® Falls” parent line plants and the “Northern Lights®” parent line plants. FIG. 6A shows an exemplary sideview of a “Beehive® White” Pentas plant that is 12 weeks old. FIG. 6B shows a top view of an exemplary “Beehive® White” Pentas plant that is 12 weeks old. FIG. 6C show a population of young “Beehive® White” Pentas plants. In FIGS. 6A-6C, it can be seen that the “Beehive®” Pentas plant does not exhibit gravitropic growth.

FIGS. 7A-7C show exemplary images of “Graffiti® Falls” parent line plants that flower red. FIG. 7A shows an exemplary top view of a “Graffiti® Falls” parent line plant. FIG. 7B shows an exemplary side view of a “Graffiti® Falls” parent line plant. FIG. 7C shows an exemplary view of multiple “Graffiti® Falls” parent line plants.

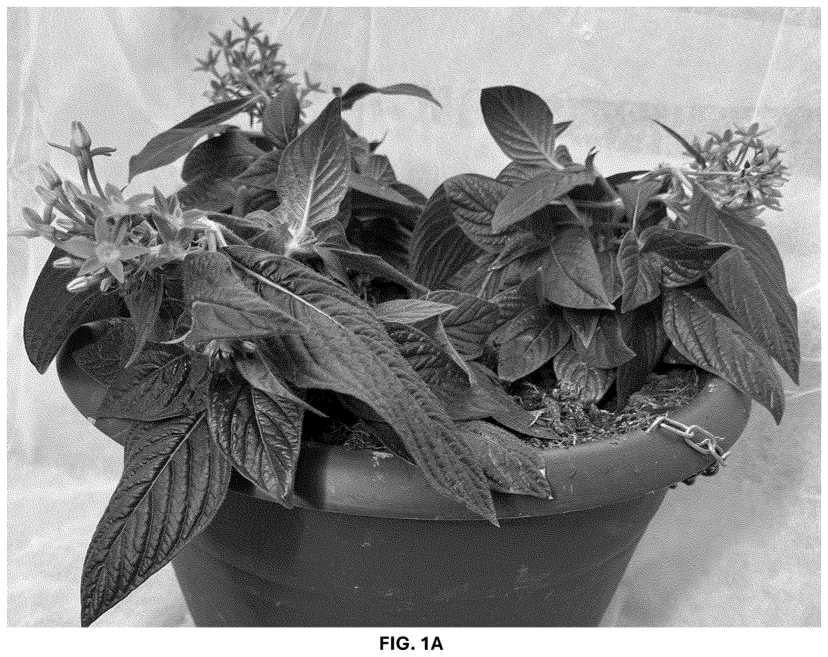

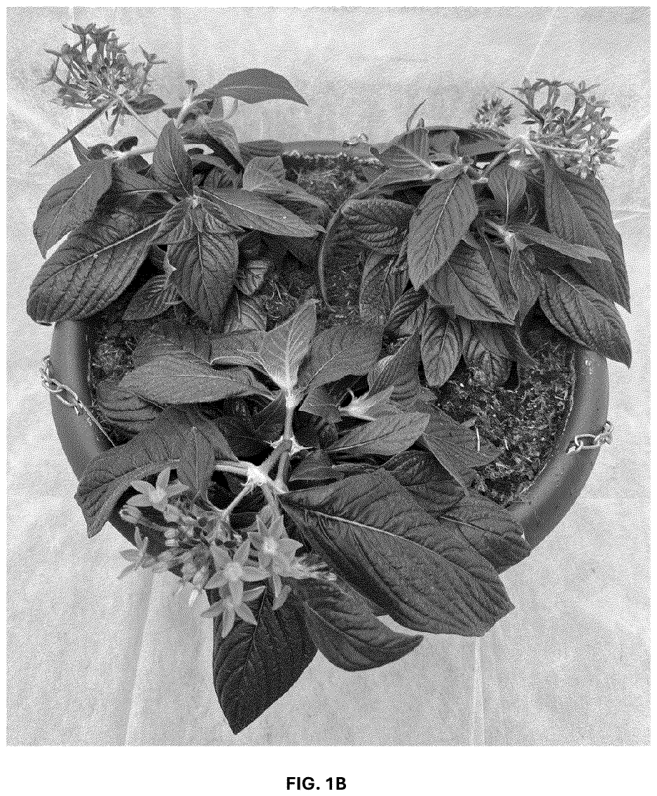

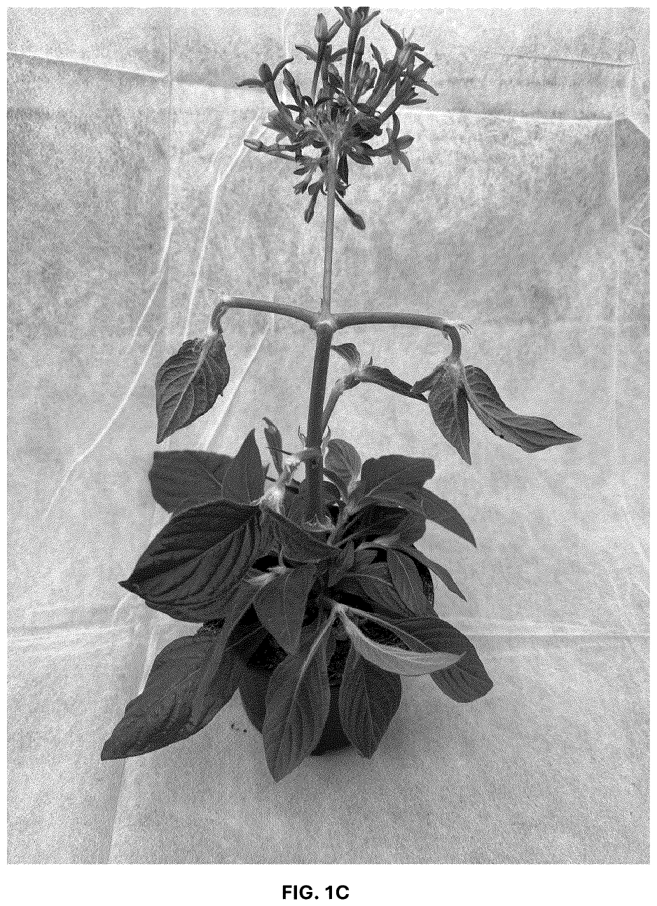

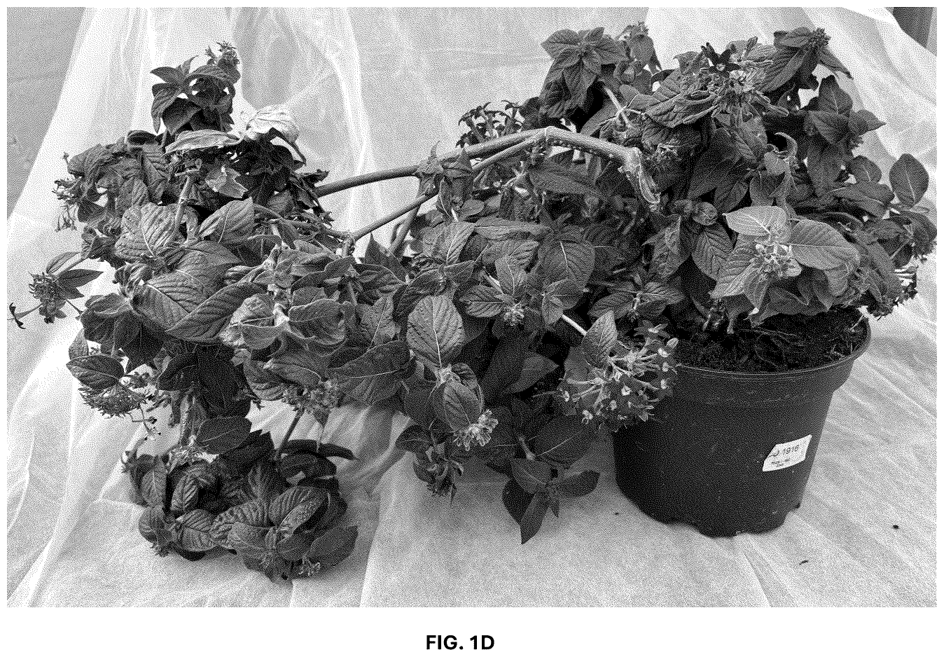

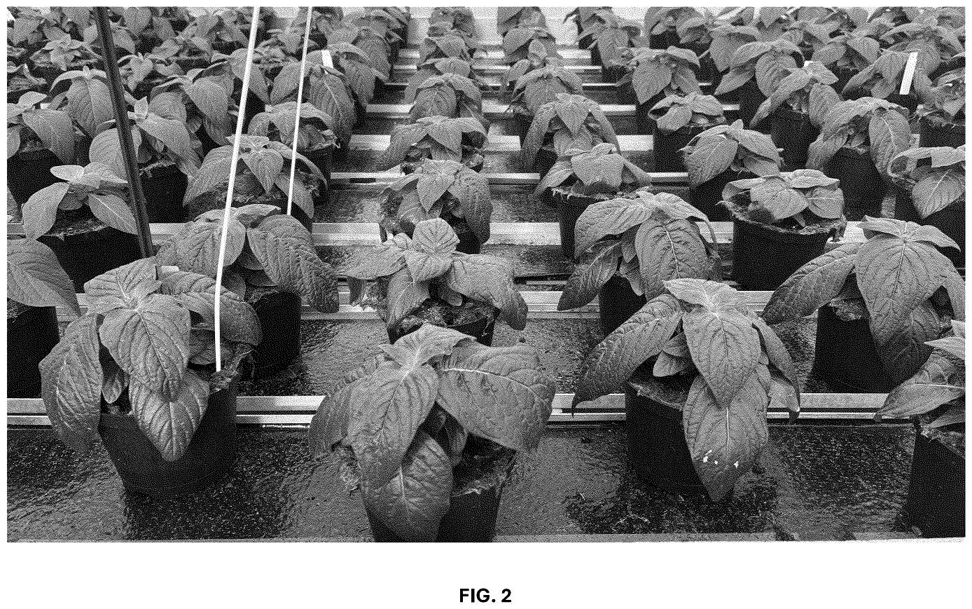

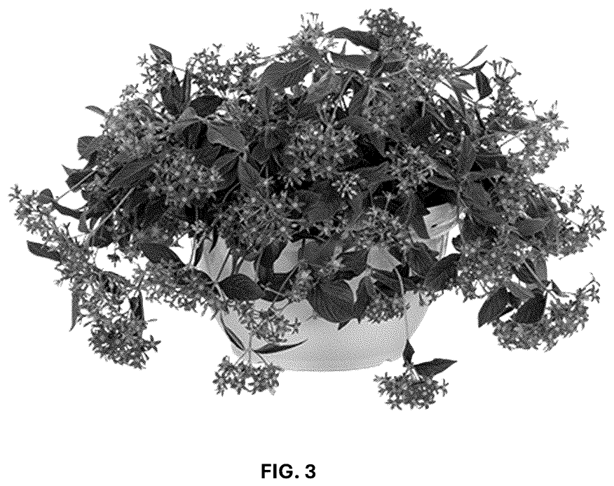

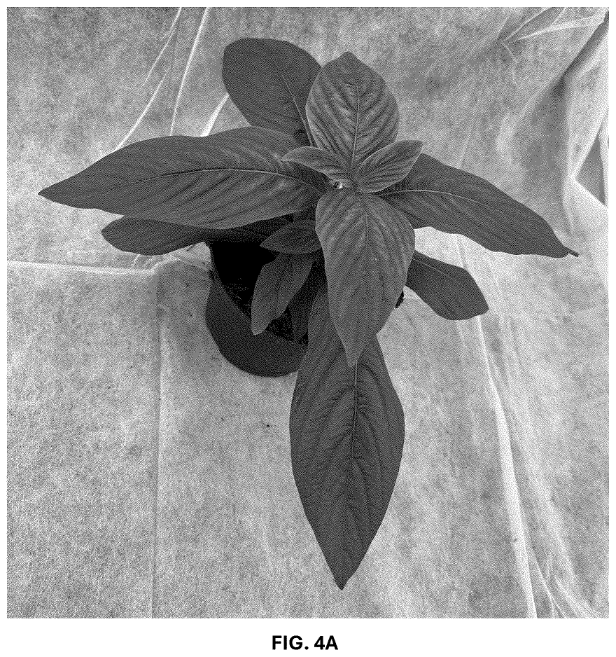

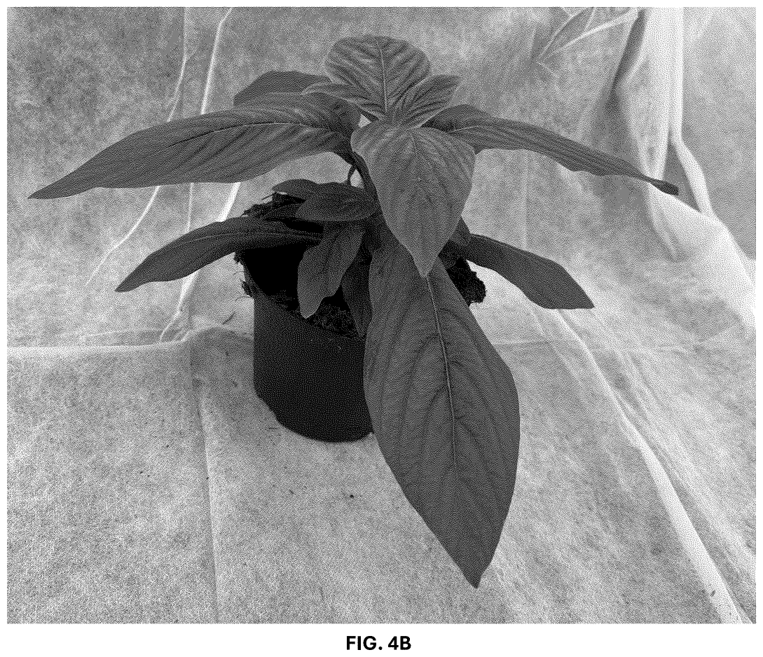

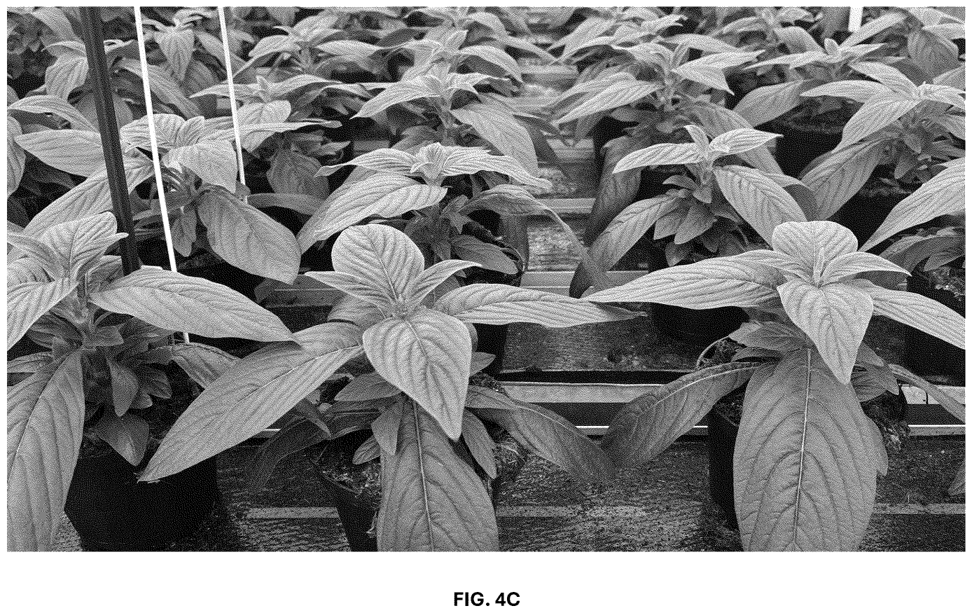

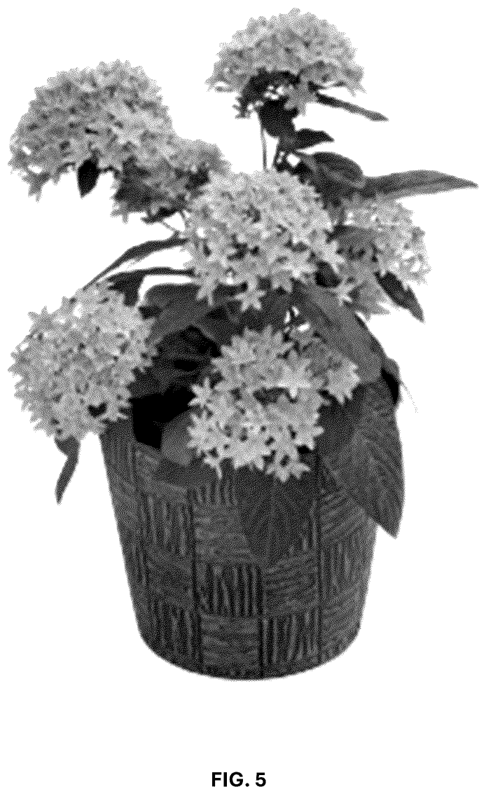

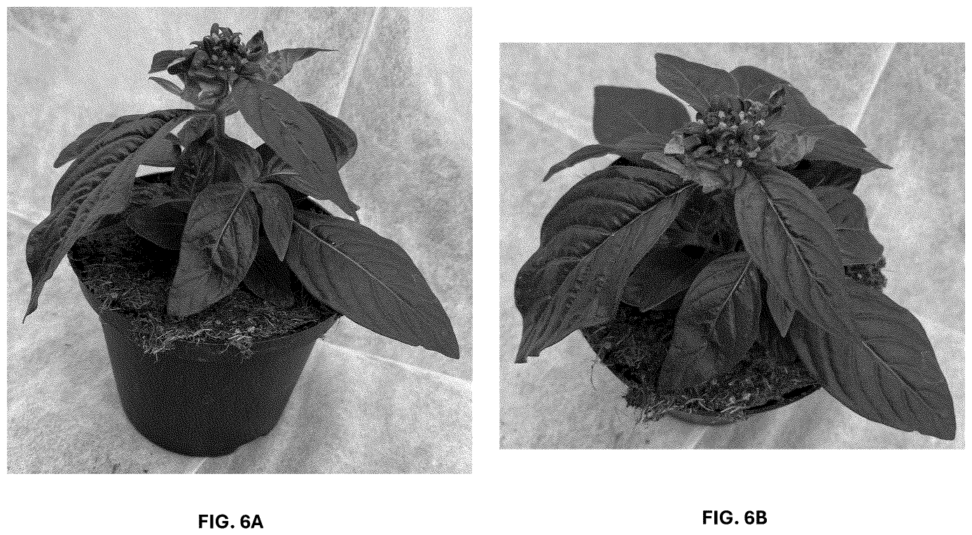

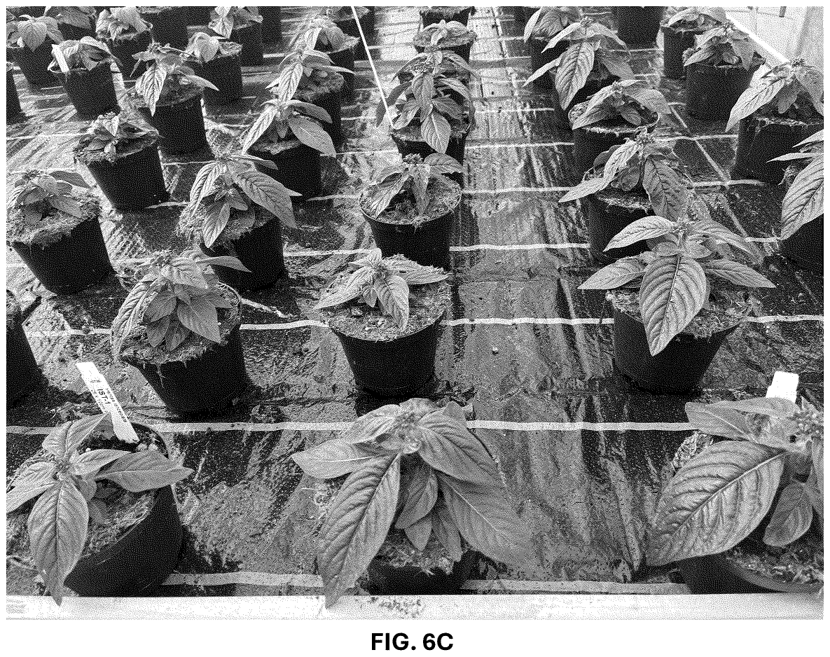

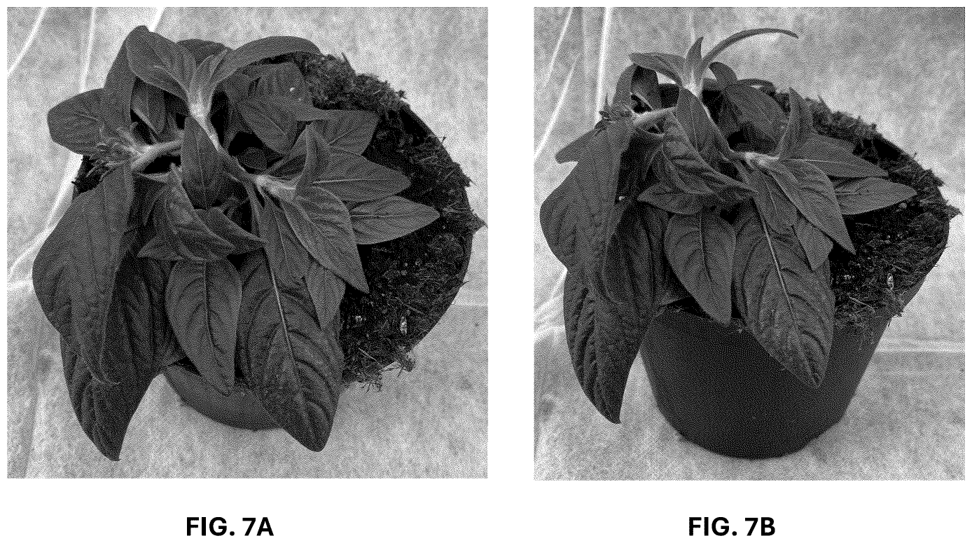

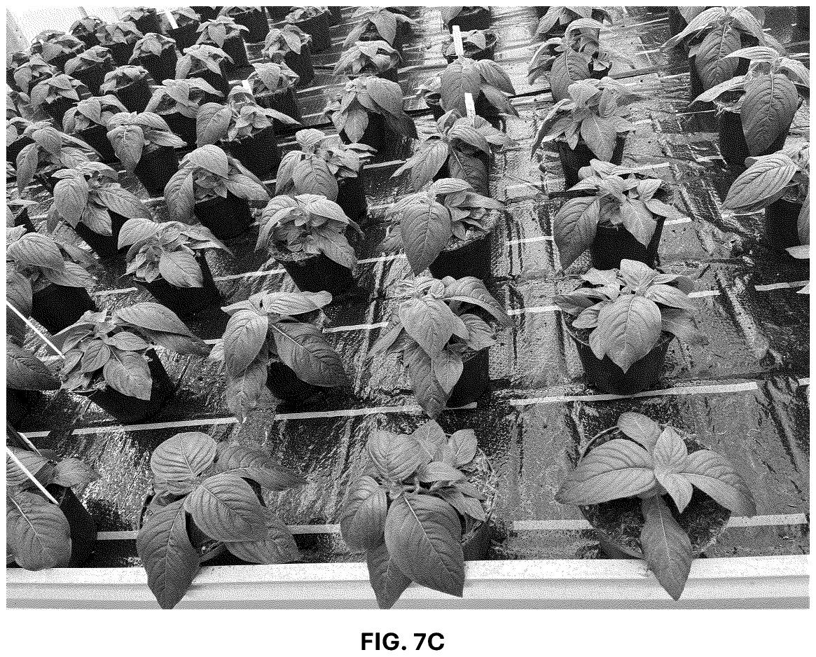

### BRIEF SUMMARY

In order to meet these needs, the present disclosure is directed to Pentas “Graffiti® Falls” parent lines and Pentas “Northern Lights®” parent lines. The gravitropic growth of “Graffiti® Falls” allows the seedlings to grow upright before the stem turns downward at angle making this variety particularly well adapted to hanging baskets.

Thus, in one aspect, the present disclosure is directed to a Pentas parent line plant comprising: (a) an gravitropic growth habit; (b) a branch shoot length of about 1 cm to about 5.5 cm; (c) a leaf width of about 3 cm to about 8 cm; (d) a flower corolla diameter of about 1.6 cm to about 2.4 cm; and (e) a stem color selected from the group consisting of RHS 143A, RHS 143B, and RHS 143C. In some embodiments, the plant further comprises a flower corolla tube color selected from the group consisting of: RHS 49D, RHS 50B, RHS 52B, RHS 53C, RHS 54D, RHS N57C, RHS 58A, RHS 61C, RHS 71A, RHS N74B, RHS 77A, RHS 77B, and RHS N79C.

In another embodiment of this aspect, the present disclosure is directed to a plant part from the plant of any one of the preceding embodiments, wherein said part is a leaf, a flower, an ovule, a pollen grain, a seed, a fruit, a cell, or a portion thereof. In some embodiments, said part is a flower.

In some embodiments, said part is a seed selected from the group consisting of: a Pentas seed designated as ‘PN25-1-2141’, representative sample of seed having been deposited under ATCC Accession Number X6; a Pentas seed designated as ‘PN25-1-2513’, representative sample of seed having been deposited under ATCC Accession Number X7; a Pentas seed designated as ‘PN25-1-2249’, representative sample of seed having been deposited under ATCC Accession Number X8; a Pentas seed designated as ‘PN25-1-2346’, representative sample of seed having been deposited under ATCC Accession Number X9; and a Pentas seed designated as ‘PN25-1-2358’, representative sample of seed having been deposited under ATCC Accession Number X10.

In a further embodiment of this aspect, the present disclosure is directed to a Pentas plant having all the physiological and morphological characteristics of the plant of any of the preceding embodiments. In another embodiment of this aspect, the present disclosure is directed to a plant part from the plant of any one of the preceding embodiments, wherein said part is a leaf, a flower, an ovule, a pollen grain, a seed, a fruit, a cell, or a portion thereof. In some embodiments, said part is a flower.

In an additional embodiment of this aspect, the present disclosure is directed to an F1 hybrid Pentas plant having a Pentas “Graffiti® Falls” parent line plant as a parent.

In one embodiment, the present disclosure is directed to a pollen grain or an ovule of the plant of any one of the preceding embodiments. In another embodiment, the present disclosure is directed to a tissue or cell culture produced from protoplasts or cells from the plant of any one of the preceding embodiments, wherein said cells or protoplasts are produced from a plant part selected from the group consisting of root, root tip, meristematic cell, stem, hypocotyl, petiole, cotyledon, leaf, flower, anther, pollen, pistil, embryo, seed, and fruit. In an additional embodiment, the present disclosure is directed to a Pentas plant regenerated from the tissue or cell culture of any of the preceding embodiments, wherein the plant has all of the morphological and physiological characteristics of a Pentas “Graffiti® Falls” parent line plant of any one of the preceding embodiment.

In a further aspect, the present disclosure is directed to a method of making Pentas seeds, said method including crossing the plant of any one of the preceding embodiments with another Pentas plant and harvesting seed therefrom. In an additional embodiment, the method further includes (a) selecting one or more of the F1 progeny for plants that have a desired trait to produce selected progeny plants; (b) crossing the selected progeny plants with a Pentas “Graffiti® Falls” parent line to produce backcross progeny plants; (c) selecting for backcross progeny plants that have the desired trait and all of the physiological and morphological characteristics of a Pentas “Graffiti® Falls” parent line when grown in the same environmental conditions to produce selected backcross progeny plants; and (d) repeating steps (b) and (c) three or more times in succession to produce selected fourth or higher backcross progeny plants that include the desired trait and all of the physiological and morphological characteristics of a Pentas “Graffiti® Falls” parent line when grown in the same environmental conditions. In some embodiments, the desired trait is herbicide resistance, insect resistance, resistance for bacterial, fungal, or viral disease, improved horticultural quality, or color.

In an additional aspect, the present disclosure is directed to a method of producing a seed of a Pentas “Graffiti® Falls” parent line-derived plant, including: (a) crossing a first Pentas “Graffiti® Falls” parent line plant with a second Pentas “Graffiti® Falls” parent line plant, whereby seed of a Pentas “Graffiti® Falls” parent line-derived plant forms. In an additional embodiment, the method further includes: (b) crossing a plant grown from the Pentas “Graffiti® Falls” parent line-derived plant seed with itself or a second Pentas “Graffiti® Falls” parent line-derived plant to yield additional Pentas “Graffiti® Falls” parent line-derived plant-derived seed; c) growing the additional Pentas “Graffiti® Falls” parent line-derived seed of step (b) to yield additional Pentas “Graffiti® Falls” parent line-derived plants; and d) repeating the crossing and growing of steps (b) and (c) for an additional 3-10 generations to generate further Pentas “Graffiti® Falls” parent line-derived plants.

In an additional aspect, the present disclosure is directed to A method of producing a seed of a Pentas parent line-derived plant, comprising: (a) crossing a first Pentas parent line plant with a second Pentas parent line plant, the first Pentas plant comprising: (i) an gravitropic growth habit; (ii) a branch shoot length of about 1 cm to about 5.5 cm; (iii) a leaf width of about 3 cm to about 8 cm; (iv) a flower corolla diameter of about 1.6 cm to about 2.4 cm; and (v) a stem color selected from the group consisting of RHS 143A, RHS 143B, and RHS 143C, whereby seed of a Pentas parent line-derived plant forms. In some embodiments, the method further comprises (b) crossing a plant grown from the Pentas parent line-derived plant seed with itself or a second Pentas parent line-derived plant to yield additional Pentas parent line-derived plant-derived seed; (c) growing the additional Pentas parent line-derived seed of step (b) to yield additional Pentas parent line-derived plants; and (d) repeating the crossing and growing of steps (b) and (c) for an additional 3-10 generations to generate further Pentas parent line-derived plants.

In another aspect, the present disclosure is directed to a Pentas seed designated as ‘PN25-1-2141’, representative sample of seed having been deposited under ATCC Accession Number X6. A further aspect of the present disclosure is directed to a Pentas plant produced by growing the seed of the preceding embodiment, wherein the Pentas plant is a Pentas “Graffiti® Falls” parent line plant. In another embodiment of this aspect, the present disclosure is directed to a plant part from the plant of any one of the preceding embodiments, wherein said part is a leaf, a flower, an ovule, a pollen grain, a seed, a fruit, a cell, or a portion thereof. In some embodiments, said part is a flower.

In a further embodiment of this aspect, the present disclosure is directed to a F1 hybrid Pentas plant having ‘PN25-1-2141’ as a parent, where ‘PN25-1-2141’ is grown from the seed of the preceding embodiments.

In another aspect, the present disclosure is directed to a Pentas seed designated as ‘PN25-1-2513’, representative sample of seed having been deposited under ATCC Accession Number X7. A further aspect of the present disclosure is directed to a Pentas plant produced by growing the seed of the preceding embodiment, wherein the Pentas plant is a Pentas “Graffiti® Falls” parent line plant. In another embodiment of this aspect, the present disclosure is directed to a plant part from the plant of any one of the preceding embodiments, wherein said part is a leaf, a flower, an ovule, a pollen grain, a seed, a fruit, a cell, or a portion thereof. In some embodiments, said part is a flower.

In a further embodiment of this aspect, the present disclosure is directed to a F1 hybrid Pentas plant having ‘PN25-1-2513’ as a parent, where ‘PN25-1-2513’ is grown from the seed of the preceding embodiments.

In another aspect, the present disclosure is directed to a Pentas seed designated as ‘PN25-1-2249’, representative sample of seed having been deposited under ATCC Accession Number X8. A further aspect of the present disclosure is directed to a Pentas plant produced by growing the seed of the preceding embodiment, wherein the Pentas plant is a Pentas “Graffiti® Falls” parent line plant. In another embodiment of this aspect, the present disclosure is directed to a plant part from the plant of any one of the preceding embodiments, wherein said part is a leaf, a flower, an ovule, a pollen grain, a seed, a fruit, a cell, or a portion thereof. In some embodiments, said part is a flower.

In a further embodiment of this aspect, the present disclosure is directed to a F1 hybrid Pentas plant having ‘PN25-1-2249’ as a parent, where ‘PN25-1-2249’ is grown from the seed of the preceding embodiments.

In another aspect, the present disclosure is directed to a Pentas seed designated as ‘PN25-1-2346’, representative sample of seed having been deposited under ATCC Accession Number X9. A further aspect of the present disclosure is directed to a Pentas plant produced by growing the seed of the preceding embodiment, wherein the Pentas plant is a Pentas “Graffiti® Falls” parent line plant. In another embodiment of this aspect, the present disclosure is directed to a plant part from the plant of any one of the preceding embodiments, wherein said part is a leaf, a flower, an ovule, a pollen grain, a seed, a fruit, a cell, or a portion thereof. In some embodiments, said part is a flower.

In a further embodiment of this aspect, the present disclosure is directed to a F1 hybrid Pentas plant having ‘PN25-1-2346’ as a parent, where ‘PN25-1-2346’ is grown from the seed of the preceding embodiments.

In another aspect, the present disclosure is directed to a Pentas seed designated as ‘PN25-1-2358’, representative sample of seed having been deposited under ATCC Accession Number X10. A further aspect of the present disclosure is directed to a Pentas plant produced by growing the seed of the preceding embodiment, wherein the Pentas plant is a Pentas “Graffiti® Falls” parent line plant. In another embodiment of this aspect, the present disclosure is directed to a plant part from the plant of any one of the preceding embodiments, wherein said part is a leaf, a flower, an ovule, a pollen grain, a seed, a fruit, a cell, or a portion thereof. In some embodiments, said part is a flower.

In a further embodiment of this aspect, the present disclosure is directed to a F1 hybrid Pentas plant having ‘PN25-1-2358’ as a parent, where ‘PN25-1-2358’ is grown from the seed of the preceding embodiments.

In one embodiment, the present disclosure is directed to a pollen grain or an ovule of the plant of any one of the preceding embodiments. In another embodiment, the present disclosure is directed to a tissue or cell culture produced from protoplasts or cells from the plant of any one of the preceding embodiments, wherein said cells or protoplasts are produced from a plant part selected from the group consisting of root, root tip, meristematic cell, stem, hypocotyl, petiole, cotyledon, leaf, flower, anther, pollen, pistil, embryo, seed, and fruit. In an additional embodiment, the present disclosure is directed to a Pentas plant regenerated from the tissue or cell culture of any of the preceding embodiments, wherein the plant has all of the morphological and physiological characteristics of a Pentas seed designated as ‘PN25-1-2141’, representative sample of seed having been deposited under ATCC Accession Number X6; all of the morphological and physiological characteristics of a Pentas seed designated as ‘PN25-1-2513’, representative sample of seed having been deposited under ATCC Accession Number X7; s all of the morphological and physiological characteristics of a Pentas seed designated as ‘PN25-1-2249’, representative sample of seed having been deposited under ATCC Accession Number X8; all of the morphological and physiological characteristics of a Pentas seed designated as ‘PN25-1-2346’, representative sample of seed having been deposited under ATCC Accession Number X9; or all of the morphological and physiological characteristics of a Pentas seed designated as ‘PN25-1-2358’, representative sample of seed having been deposited under ATCC Accession Number X10.

In an additional aspect, the present disclosure is directed to a method of making Pentas seeds, said method including crossing the plant of any of the previous embodiments with another Pentas plant and harvesting seed therefrom. In some embodiments, the method further includes: (a) selecting one or more of the F1 progeny for plants that have a desired trait to produce selected progeny plants; (b) crossing the selected progeny plants with a ‘PN25-1-2141’ plant, representative sample of seed having been deposited under ATCC Accession Number X6, a ‘PN25-1-2513’ plant, representative sample of seed having been deposited under ATCC Accession Number X7, a ‘PN25-1-2249’ plant, representative sample of seed having been deposited under ATCC Accession Number X8, a ‘PN25-1-2346’ plant, representative sample of seed having been deposited under ATCC Accession Number X9, or a ‘PN25-1-2358’ plant, representative sample of seed having been deposited under ATCC Accession Number X10, to produce backcross progeny plants; (c) selecting for backcross progeny plants that have the desired trait and all of the physiological and morphological characteristics of a ‘PN25-1-2141’ plant, a ‘PN25-1-2513’ plant, a ‘PN25-1-2249’ plant, a ‘PN25-1-2346’ plant, or a ‘PN25-1-2358’ plant when grown in the same environmental conditions to produce selected backcross progeny plants; and (d) repeating steps (b) and (c) three or more times in succession to produce selected fourth or higher backcross progeny plants that include the desired trait and all of the physiological and morphological characteristics of a ‘PN25-1-2141’ plant, a ‘PN25-1-2513’ plant, a ‘PN25-1-2249’ plant, a ‘PN25-1-2346’ plant, or a ‘PN25-1-2358’ plant when grown in the same environmental conditions. In some embodiments, the desired trait is herbicide resistance, insect resistance, resistance for bacterial, fungal, or viral disease, improved horticultural quality, or color.

In one aspect, the present disclosure is directed to a Pentas “Northern Lights®” parent line plant including a flower color characteristic selected from the group consisting of RHS N57D (pink/violet color group), RHS N74A (violet/pink color group), RHS N66A (red/pink color group), and red/violet color group, and a plant height characteristic selected from the group consisting of extreme dwarf, dwarf, medium, and tall. In some embodiments, the plant includes the flower color characteristic RHS N57D (pink/violet color group). In some embodiments, the plant includes the flower color characteristic RHS N74A (violet/pink color group). In some embodiments, the plant includes the flower color characteristic RHS N66A (red/pink color group). In some embodiments, the plant includes the flower color characteristic red/violet color group.

In another embodiment of this aspect, the present disclosure is directed to a plant part from the plant of any one of the preceding embodiments, wherein said part is a leaf, a flower, an ovule, a pollen grain, a seed, a fruit, a cell, or a portion thereof. In some embodiments, said part is a flower.

In a further embodiment of this aspect, the present disclosure is directed to a Pentas plant having all the physiological and morphological characteristics of the plant of any of the preceding embodiments. In another embodiment of this aspect, the present disclosure is directed to a plant part from the plant of any one of the preceding embodiments, wherein said part is a leaf, a flower, an ovule, a pollen grain, a seed, a fruit, a cell, or a portion thereof. In some embodiments, said part is a flower.

In an additional embodiment of this aspect, the present disclosure is directed to an F1 hybrid Pentas plant having a Pentas “Northern Lights®” parent line plant as a parent.

In one embodiment, the present disclosure is directed to a pollen grain or an ovule of the plant of any one of the preceding embodiments. In another embodiment, the present disclosure is directed to a tissue or cell culture produced from protoplasts or cells from the plant of any one of the preceding embodiments, wherein said cells or protoplasts are produced from a plant part selected from the group consisting of root, root tip, meristematic cell, stem, hypocotyl, petiole, cotyledon, leaf, flower, anther, pollen, pistil, embryo, seed, and fruit. In an additional embodiment, the present disclosure is directed to a Pentas plant regenerated from the tissue or cell culture of any of the preceding embodiments, wherein the plant has all of the morphological and physiological characteristics of a Pentas “Northern Lights®” parent line plant of any one of the preceding embodiment.

In a further aspect, the present disclosure is directed to a method of making Pentas seeds, said method including crossing the plant of any one of the preceding embodiments with another Pentas plant and harvesting seed therefrom. In an additional embodiment, the method further includes (a) selecting one or more of the F1 progeny for plants that have a desired trait to produce selected progeny plants; (b) crossing the selected progeny plants with a Pentas “Northern Lights®” parent line to produce backcross progeny plants; (c) selecting for backcross progeny plants that have the desired trait and all of the physiological and morphological characteristics of a Pentas “Northern Lights®” parent line when grown in the same environmental conditions to produce selected backcross progeny plants; and (d) repeating steps (b) and (c) three or more times in succession to produce selected fourth or higher backcross progeny plants that include the desired trait and all of the physiological and morphological characteristics of a Pentas “Northern Lights®” parent line when grown in the same environmental conditions. In some embodiments, the desired trait is herbicide resistance, insect resistance, resistance for bacterial, fungal, or viral disease, improved horticultural quality, or color.

In an additional aspect, the present disclosure is directed to a method of producing a seed of a Pentas “Northern Lights®” parent line-derived plant, including: (a) crossing a first Pentas “Northern Lights®” parent line plant with a second Pentas “Northern Lights®” parent line plant, whereby seed of a Pentas “Northern Lights®” parent line-derived plant forms. In an additional embodiment, the method further includes: (b) crossing a plant grown from the Pentas “Northern Lights®” parent line-derived plant seed with itself or a second Pentas “Northern Lights®” parent line-derived plant to yield additional Pentas “Northern Lights®” parent line-derived plant-derived seed; c) growing the additional Pentas “Northern Lights®” parent line-derived seed of step (b) to yield additional Pentas “Northern Lights®” parent line-derived plants; and d) repeating the crossing and growing of steps (b) and (c) for an additional 3-10 generations to generate further Pentas “Northern Lights®” parent line-derived plants.

In another embodiment of this aspect, the present disclosure is directed to a plant part from the plant of any one of the preceding embodiments, wherein said part is a leaf, a flower, an ovule, a pollen grain, a seed, a fruit, a cell, or a portion thereof. In some embodiments, said part is a flower.

In some embodiments, said part is a seed selected from the group consisting of: a Pentas seed designated as ‘PN25-1-1959’, representative sample of seed having been deposited under ATCC Accession Number X1; a Pentas seed designated as ‘PN25-1-1965’, representative sample of seed having been deposited under ATCC Accession Number X3; a Pentas seed designated as ‘PN25-1-1968’, representative sample of seed having been deposited under ATCC Accession Number X4; and a Pentas seed designated as ‘PN25-1-1973’, representative sample of seed having been deposited under ATCC Accession Number X5.

In a further embodiment of this aspect, the present disclosure is directed to a Pentas plant having all the physiological and morphological characteristics of the plant of any of the preceding embodiments. In another embodiment of this aspect, the present disclosure is directed to a plant part from the plant of any one of the preceding embodiments, wherein said part is a leaf, a flower, an ovule, a pollen grain, a seed, a fruit, a cell, or a portion thereof. In some embodiments, said part is a flower.

In a further embodiment of this aspect, the present disclosure is directed to a F1 hybrid Pentas plant having ‘PN25-1-1959’ as a parent, where ‘PN25-1-1959’ is grown from the seed of the preceding embodiments; having ‘PN25-1-1965’ as a parent, where ‘PN25-1-1965’ is grown from the seed of the preceding embodiments; having ‘PN25-1-1968’ as a parent, where ‘PN25-1-1968’ is grown from the seed of the preceding embodiments; or having ‘PN25-1-1973’ as a parent, where ‘PN25-1-1973’ is grown from the seed of the preceding embodiments.

In one embodiment, the present disclosure is directed to a pollen grain or an ovule of the plant of any one of the preceding embodiments. In another embodiment, the present disclosure is directed to a tissue or cell culture produced from protoplasts or cells from the plant of any one of the preceding embodiments, wherein said cells or protoplasts are produced from a plant part selected from the group consisting of root, root tip, meristematic cell, stem, hypocotyl, petiole, cotyledon, leaf, flower, anther, pollen, pistil, embryo, seed, and fruit. In an additional embodiment, the present disclosure is directed to a Pentas plant regenerated from the tissue or cell culture of any of the preceding embodiments, wherein the plant has all of the morphological and physiological characteristics of a Pentas plant produced by growing a Pentas seed designated as ‘PN25-1-1959’, representative sample of seed having been deposited under ATCC Accession Number X1; all of the morphological and physiological characteristics of a Pentas seed designated as ‘PN25-1-1965’, representative sample of seed having been deposited under ATCC Accession Number X3; all of the morphological and physiological characteristics of a Pentas seed designated as ‘PN25-1-1968’, representative sample of seed having been deposited under ATCC Accession Number X4; or all of the morphological and physiological characteristics of a Pentas seed designated as ‘PN25-1-1973’, representative sample of seed having been deposited under ATCC Accession Number X5.

In an additional aspect, the present disclosure is directed to a method of making Pentas seeds, said method including crossing the plant of any of the previous embodiments with another Pentas plant and harvesting seed therefrom. In some embodiments, the method further includes: (a) selecting one or more of the F1 progeny for plants that have a desired trait to produce selected progeny plants; (b) crossing the selected progeny plants with a ‘PN25-1-1959’ plant, representative sample of seed having been deposited under ATCC Accession Number X1; a ‘PN25-1-1965’ plant, representative sample of seed having been deposited under ATCC Accession Number X3; a ‘PN25-1-1968’ plant, representative sample of seed having been deposited under ATCC Accession Number X4; or a ‘PN25-1-1973’ plant, representative sample of seed having been deposited under ATCC Accession Number X5, to produce backcross progeny plants; (c) selecting for backcross progeny plants that have the desired trait and all of the physiological and morphological characteristics of a ‘PN25-1-1959’ plant, a ‘PN25-1-1965’ plant, a ‘PN25-1-1968’ plant, or a ‘PN25-1-1973’ plant when grown in the same environmental conditions to produce selected backcross progeny plants; and (d) repeating steps (b) and (c) three or more times in succession to produce selected fourth or higher backcross progeny plants that include the desired trait and all of the physiological and morphological characteristics of aa ‘PN25-1-1959’ plant, a ‘PN25-1-1965’ plant, a ‘PN25-1-1968’ plant, or a ‘PN25-1-1973’ plant when grown in the same environmental conditions. In some embodiments, the desired trait is herbicide resistance, insect resistance, resistance for bacterial, fungal, or viral disease, improved horticultural quality, or color.

In a further embodiment of this aspect, the present disclosure is directed to a Pentas plant having all the physiological and morphological characteristics of the plant of any of the preceding embodiments. In another embodiment of this aspect, the present disclosure is directed to a plant part from the plant of any one of the preceding embodiments, wherein said part is a leaf, a flower, an ovule, a pollen grain, a seed, a fruit, a cell, or a portion thereof. In some embodiments, said part is a flower.

In addition to the exemplary aspects and embodiments described above, further aspects and embodiments will become apparent by reference by study of the following descriptions.

### DETAILED DESCRIPTION

There are numerous steps in the development of any novel, desirable plant germplasm. Plant breeding begins with the analysis and definition of problems and weaknesses of the current germplasm, the establishment of program goals, and the definition of specific breeding objectives. The next step is selection of germplasm that possess the traits to meet the program goals. The goal is to combine in a single variety or hybrid an improved combination of desirable traits from the parental germplasm. These important traits may include improved flower color, gravitropic growth, attractive growth habit, and better horticultural quality.

### Definitions

In the description and tables that follow, a number of terms are used. In order to provide a clear and consistent understanding of the specification and claims, including the scope to be given such terms, the following definitions are provided:

Allele. The allele is any of one or more alternative forms of a gene, all of which relate to one trait or characteristic. In a diploid cell or organism, the two alleles of a given gene occupy corresponding loci on a pair of homologous chromosomes.

Alter. The utilization of up-regulation, down-regulation, or gene silencing.

Gravitropic growth. As used herein, the shoots grow upwards before turning and growing downwards towards the earth, which is the opposite of normal negative gravitropic growth displayed by shoots.

Backcrossing. Backcrossing is a process in which a breeder repeatedly crosses hybrid progeny back to one of the parents, for example, a first generation hybrid F1 with one of the parental genotype of the F1 hybrid. Backcrossing is also a process in which a breeder crosses hybrid progeny to a recurrent species or plant phenotype.

Cell. Cell as used herein includes a plant cell, whether isolated, in tissue culture or incorporated in a plant or plant part.

Clone. A plant or a group of plants that is propagated vegetatively from a single plant.

Cotyledon. One of the first leaves of the embryo of a seed plant; typically one or more in monocotyledons, two in dicotyledons, and two or more in gymnosperms.

Embryo. The embryo is the small plant contained within a mature seed.

Essentially all the physiological and morphological characteristics. A plant having essentially all the physiological and morphological characteristics means a plant having the physiological and morphological characteristics of the recurrent parent, except for the characteristics derived from the converted gene.

F #. The “F” symbol denotes the filial generation, and the # is the generation number, such as F₁, F₂, F₃, etc.

Genetically Modified. Describes an organism that has received genetic material from another, or had its genetic material modified, resulting in a change in one or more of its phenotypic characteristics. Methods used to modify, introduce or delete the genetic material may include mutation breeding, backcross conversion, genetic transformation, single and multiple gene conversion, and/or direct gene transfer.

Gene Silencing. The interruption or suppression of the expression of a gene at the level of transcription or translation.

Genotype. Refers to the genetic constitution of a cell or organism.

Hypocotyl. A hypocotyl is the portion of an embryo or seedling between the cotyledons and the root. Therefore, it can be considered a transition zone between shoot and root.

Linkage. Refers to a phenomenon wherein alleles on the same chromosome tend to segregate together more often than expected by chance if their transmission was independent.

Linkage Disequilibrium. Refers to a phenomenon wherein alleles tend to remain together in linkage groups when segregating from parents to offspring, with a greater frequency than expected from their individual frequencies.

Locus. A locus confers one or more traits such as, for example, herbicide tolerance, insect resistance, disease resistance, waxy starch, modified fatty acid metabolism, modified phytic acid metabolism, modified carbohydrate metabolism, modified protein metabolism. The trait may be, for example, conferred by a naturally occurring gene introduced into the genome of the variety by backcrossing, a natural or induced mutation, or a transgene introduced through genetic transformation techniques. A locus may comprise one or more alleles integrated at a single chromosomal location.

Multiple Gene Converted (Conversion). Multiple gene converted (conversion) includes plants developed by a plant breeding technique called backcrossing wherein essentially all of the desired morphological and physiological characteristics of an inbred are recovered, while retaining two or more genes transferred into the inbred via crossing and backcrossing. The term can also refer to the introduction of multiple genes through genetic engineering techniques known in the art.

Pedigree Distance. Relationship among generations based on their ancestral links as evidenced in pedigrees. Genetic similarity decreases with pedigree distance. May be measured by the distance of the pedigree from a given starting point in the ancestry.

Percent Identity. Percent identity as used herein refers to the comparison of the alleles of two Pentas varieties. Percent identity is determined by comparing a statistically significant number of the alleles of two developed varieties. For example, a percent identity of 90% between Pentas variety 1 and Pentas variety 2 means that the two varieties have the same allele at 90% of their loci.

Percent Similarity. Percent similarity as used herein refers to the comparison of the homozygous alleles of a Pentas parent line with another plant, and if the homozygous allele of parent line matches at least one of the alleles from the other plant then they are scored as similar. Percent similarity is determined by comparing a statistically significant number of loci and recording the number of loci with similar alleles as a percentage. A percent similarity of 90% between 99/280-6 and another plant means that 99/280-6 matches at least one of the alleles of the other plant at 90% of the loci.

Plant. As used herein, the term “plant” includes reference to an immature or mature whole plant, including a plant from which seed or grain or anthers have been removed. Seed or embryo that will produce the plant is also considered to be the plant.

Plant Height. Plant height is taken from the top of the soil to the top node of the plant and is measured in centimeters.

Plant Parts. As used herein, the term “plant parts” (or a Pentas plant, or a part thereof) includes but is not limited to protoplasts, leaves, stems, roots, root tips, anthers, pistils, seed, embryo, pollen, ovules, cotyledon, hypocotyl, pod, flower, shoot, tissue, petiole, cells, meristematic cells, fruit, portions thereof, and the like.

Plant Tissue. As used herein, the term “plant tissue” means tissue from a plant part, for example protoplasts, leaves, stems, roots, root tips, anthers, seed, embryo, pollen, ovules, cotyledon, hypocotyl, flower, shoot, tissue, petiole, cells, meristematic cells and any other plant part.

Quantitative Trait Loci (QTL). As used herein, “quantitative trait loci” refers to genetic loci that control to some degree numerically representable traits that are usually continuously distributed.

Regeneration. As used herein, “regeneration” refers to the development of a plant from tissue culture.

RHS. RHS refers to the Royal Horticultural Society of England which publishes an official botanical color chart quantitatively identifying colors according to a defined numbering system. The chart may be purchased from Royal Horticulture Society Enterprise Ltd., RHS Garden; Wisley, Woking; Surrey GU236QB, UK.

Single Gene Converted. Single gene converted (conversion) plants refers to plants which are developed by a plant breeding technique called backcrossing or via genetic engineering wherein essentially all of the desired morphological and physiological characteristics of a line are recovered in addition to the single gene transferred into the line via the backcrossing technique or via genetic engineering.

Trailing growth habit. A growth habit that is characterized by growing outwards and/or downwards.

Vegetatively propagating. Means to produce a plant or plant part asexually from a single plant or plant part.

### Overview of the Pentas “Graffiti® Falls” Parent Lines

Pentas “Graffiti® Falls” parent lines differ from currently available Pentas varieties by having an gravitropic growth habit. Pentas “Graffiti® Falls” parent lines are the result of numerous generations of plant selections chosen for their gravitropic growth habit.

Pentas “Graffiti® Falls” parent lines have shown uniformity and stability, as described in the following “Objective Description of Pentas “Graffiti® Falls” Parent Lines” section. They have been self-pollinated a sufficient number of generations with careful attention to uniformity of plant type. The lines have been increased with continued attention to confirm transference, heritability, and stable performance as hybrid parents.

There are twenty-one Pentas “Graffiti® Falls” parent lines that share the common parent Pentas clone ‘99/280-6’ (U.S. Pat. No. 7,880,073), a variety having increased cold tolerance (e.g., as low as 4.0° C.), increased flowering, and unique inflorescence. Table 1 provides the Pentas “Graffiti® Falls” parent line designations and pedigrees. Column 1 provides the general identifier used for the parent line pedigree, which corresponds to the “Parent line designation” column in Table 5 of U.S. Provisional Application No. 63/775,505. If multiple lines have the same pedigree, this is indicated by an additional number after a period; for example GF2.1, GF2.2, and GF2.3 indicate three lines with the shared pedigree Attmarinmar/Albert1. Column 2 provides the parent line designation for each Pentas “Graffiti® Falls” parent line. Column 3 provides the pedigree for each parent line. As noted above, Pentas clone ‘99/280-6’ (U.S. Pat. No. 7,880,073) is a shared parent in all pedigrees, and is also known as “99/280-6” or “Att”. The line “Attmarinmar” corresponds to ((99/280-6×Mauritius) (Indira×Mauritius)), which was presented in U.S. Provisional Application No. 63/775,505 as ((99/280-6×mar) (in×mar)).

TABLE 1
Pentas “Graffiti ® Falls” parent lines and pedigrees.

| General | Exemplary | Parent line | |
|---|---|---|---|
| identifier | plant | designation | Parent line pedigree |
| GF1 | PN25-1- | PN25-1-2141 | (Hans T17 T17 × (Attmarinmar¹ × Albert1)) |
| | 1773-2141 | | |
| GF2.1 | PN25-1- | PN25-1-2144 | Attmarinmar/Albert1 |
| | 1775-2144 | | |
| GF7 | PN25-1- | PN25-1-2166 | (Attmarinmar × Albert1) |
| | 1799-2166 | | |
| GF11.1 | PN25-1- | PN25-1-2196 | ((Heinz/Siegfried × Baldo/P. speciosa) × |
| | 1831-2196 | | Attmarinmar/Albert1) |
| GF11.2 | PN25-1- | PN25-1-2196 | ((Heinz/Siegfried × Baldo/P. speciosa) × |
| | 1834-2196 | | Attmarinmar/Albert1) |
| GF18 | PN25-1- | PN25-1-2223 | (((Attmarinmar × Albert1) × (Northern Lights × |
| | 1854-2223 | | Hans T17)) × (Heinz/Siegfried × |
| | | | Baldo/P. speciosa)) |
| GF21 | PN25-1- | PN25-1-2243 | (((Northern Lights × Hans T17) × (Attmarinmar × |
| | 1878-2243 | | Albert1)) × Heinz) |
| GF22.1 | PN25-1- | PN25-1-2249 | (((Northern Lights × Hans T17) × (Attmarinmar × |
| | 1882-2249 | | Albert1)) × (Heinz × Cowei/Admiral)) |
| GF22.2 | PN25-1- | PN25-1-2367 | (((Northern Lights × Hans T17) × (Attmarinmar × |
| | 2028-2367 | | Albert1)) × (Heinz × Cowei/Admiral)) |
| GF28 | PN25-1- | PN25-1-2346 | (((Attmarinmar × Albert1) × (Felix × (99/280- |
| | 2007-2346 | | 6/P. speciosa × (Attmarinmar × Albert1)))) × |
| | | | BeeBright Red) |
| GF33.1 | PN25-1- | PN25-1-2358 | (Attmarinmar/Albert1 × (Hans/Felix × |
| | 2012-2358 | | Attmarinmar)) |
| GF33.2 | PN25-1- | PN25-1-2358 | (Attmarinmar/Albert1 × (Hans/Felix × |
| | 2013-2358 | | Attmarinmar)) |
| GF34.1 | PN25-1- | PN25-1-2434 | (((Attmarinmar × Albert1) × (Northern Lights × |
| | 2103-2434 | | Hans T17)) × (Heinz × Indira/99/280-6)) |
| GF34.2 | PN25-1- | PN25-1-2451 | (((Attmarinmar × Albert1) × (Northern Lights × |
| | 2123-2451 | | Hans T17)) × (Heinz × Indira/99/280-6)) |
| GF34.3 | PN25-1- | PN25-1-2456 | (((Attmarinmar × Albert1) × (Northern Lights × |
| | 2125-2456 | | Hans T17)) × (Heinz × Indira/99/280-6)) |
| GF34.4 | PN25-1- | PN25-1-2456 | (((Attmarinmar × Albert1) × (Northern Lights × |
| | 2126-2456 | | Hans T17)) × (Heinz × Indira/99/280-6)) |
| GF34.5 | PN25-1- | PN25-1-2459 | (((Attmarinmar × Albert1) × (Northern Lights × |
| | 2130-2459 | | Hans T17)) × (Heinz × Indira/99/280-6)) |
| GF35.1 | PN25-1- | PN25-1-2473 | (((Latona/Felix × Rota.Attmarinmar) × Hans T17 |
| | 2147-2473 | | mar) × (Northern Lights × Hans T17)) |
| GF35.2 | PN25-1- | PN25-1-2509 | (((Latona/Felix × Rota.Attmarinmar) × Hans T17 |
| | 2165-2509 | | mar) × (Northern Lights × Hans T17)) |
| GF35.3 | PN25-1- | PN25-1-2509 | (((Latona/Felix × Rota.Attmarinmar) × Hans T17 |
| | 2167-2509 | | mar) × (Northern Lights × Hans T17)) |
| GF36 | PN25-1- | PN25-1-2513 | (((Attmarinmar × Albert1) × (Felix × 99/280- |
| | 2180-2513 | | 6/P. speciosa)) × ((Hans T17 T17 × |
| | | | Bonny/Baldo) × Viola/Rosaa.Attmarinmar)) |

¹Attmarinmar is (((99/280-6 × Mauritius) (Indira × Mauritius))

### Objective Description of Pentas “Graffiti® Falls” Parent Lines

Pentas “Graffiti® Falls” parent lines have the following morphologic and other characteristics. These characteristics are based primarily on data collected in greenhouses where the plants are given adequate space to grow freely. Adequate space suitable for all “Graffiti® Falls” parent lines is approximately 7 inches center to center. A minimum of 4 to 5 inches center to center is needed to develop the habit correctly. When grown too close to each other, the plants may be influenced by the other plant to grow up and thus inhibit their true growth habit. Careful monitoring for habit and adjustment of spacing as needed is recommended. Color references are primarily to The RHS Colour Chart of The Royal Horticultural Society of London (RHS) (5th edition). The below characteristics are characteristics shared by all Pentas “Graffiti® Falls” parent lines. Where characteristics vary depending on the line, this is indicated as “line-specific”. For “line-specific” characteristics, see the “Objective Description of Pentas “Graffiti® Falls” Parent Lines Line-specific Characteristics” section below.

### Classification:

Family: Rubiaceae
Botanical name: Pentas sp.
Common name: Pentas

### Plant:

Life cycle: Used as an annual horticulturally
Habit: Upward growth followed by gravitropic growth and outward growth (i.e.,
trailing growth) is line-specific
Height: 18 to 60 cm; line specific.
Width: 34 to 79 cm; line specific

### Stems:

Anthocyanin color: Absent or present (Strong Yellow Green, RHS 143A and RHS144A); line-specific
Stem length: 9 cm to 29 cm; line-specific
Stem internodes: 4 mm to 3 cm; line specific

### Leaves:

Arrangement: In pairs in an opposite arrangement up the stem; each leaf has an auxiliary bud that may develop into a lateral shoot
Shape: Between lanceolate and ovate
Apex: Pointed
Base: Tapering
Margin: Entire
Size:
   Length: In average 4 cm to 12 cm, up to 15 cm
   Width: 3 cm to 6 cm up to 8 cm
Venation: Smooth, not pronounced on surface

### Flowers:

Inflorescence:
   Type: Pendulate cyme (hanging flower clusters)
   Diameter: Highly variable. In the bud stage, the flowers are 2 cm to 3 cm across. While flowering, the flowers can be 5 cm to 10 cm in diameter.
Number of flowers: 15 to 60; highly variable
Flower opening: 2 to 20 open at a time; highly variable
Fragrance: None
Corolla:
   Shape: 5 tubular petals, long tube ending in a 5 pointed star, the pale pistil is a bit longer than the flower, anthers are positioned visibly in the tube just below the upper end and at this point the tube is a little bit wider
   Diameter: 1.6 cm to 2.4 cm
   Depth: 1.8 cm to 4 cm
   Tube diameter: 2 mm to 4 mm
   Tube color: RHS 49D (Light red pink), RHS 50B (Dark pink red), RHS 52B (Red pink), RHS 53C (Dark pink red), RHS 54D (Light pink), RHS N57C (Dark pink), RHS 58A (Purple), RHS 61C (Dark pink), RHS 71A (Purple), RHS N74B (Purple pink), RHS 77A (Dark pink violet), RHS 77B (Dark pink violet), or RHS N79C (Purple); line-specific
Petals:
   Texture: Smooth, glossy, lower side with a slight keel
   Size: Crown diameter 1.6 to 2.5 cm
   Length: About 0.6 to 1 cm
   Width: About 4 mm to 6 mm
   Shape: Pointed, tapering
   Apex: Pointed
   Margin: Entire
Calyx (sepals):
   Shape: 5 very irregular sepals with variable length, 2 to 3 are much bigger, the others are very small and shriveled; lanceolate, quite similar to leaves.
   Apex: Pointed
   Margin: Entire
   Color of upper surface: RHS 137A (Moderate olive green)
   Color of lower surface: RHS 137B (Moderate olive green) and RHS 137C
   (Moderate yellow green)
Pedicel:
   Length: About 1 mm to 2 mm
   Diameter: About 1 mm

### Objective Description of Pentas “Graffiti® Falls” Parent Lines Line-Specific Characteristics

The below characteristics are line-specific to particular Pentas “Graffiti® Falls” parent lines. Each characteristic is uniform and stable within the line where it is present. In other words, each Pentas “Graffiti® Falls” parent line is true to type for its specific characteristic. Table 2 provides line-specific plant and stem characteristics. Vigor, as described in Table 2, is a trait generally reflected in the height and width of a plant, and can also be considered a rough estimation of overall plant mass. For example, a mid vigor plant falls in the middle of the size range, whereas a high vigor plant would be at the upper end of the size range. Table 3 provides line-specific stem characteristics, and Table 4 provides line-specific flower characteristics.

TABLE 2
Pentas “Graffiti ® Falls” plant characteristics

| Paren- | | Plant | Plant | |
|---|---|---|---|---|
| tal | | Height | width | |
| Line | Plant Habit | (cm) | (cm) | Plant Spread |
| GF1 | trailing, wider than tall | 29 | 51 | mid vigor |
| GF2.1 | trailing, wider than tall | 18 | 39 | mid vigor |
| GF7 | trailing, wider than tall | 34 | 57 | mid vigor |
| GF11.1 | mounded, wider than tall | 37 | 47 | mid vigor |
| GF11.2 | upright, little spread | 43 | 45 | Northern Lights, |
| | | | | mid vigor |
| GF18 | mounded, wider than tall | 50 | 61 | high vigor |
| GF21 | trailing, wider than tall | 38 | 51 | mid vigor |
| GF22.1 | trailing, wider than tall | 60 | 73 | high vigor |
| GF22.2 | trailing, wider than tall | 38 | 46 | high vigor |
| GF28 | trailing, wider than tall | 34 | 53 | mid vigor |
| GF33.1 | mounded, wider than tall | 48 | 60 | high vigor |
| GF33.2 | mounded, wider than tall | 47 | 54 | high vigor |
| GF34.1 | mounded, wider than tall | 42 | 54 | mid vigor |
| GF34.2 | upright, little spread | 42 | 44 | mid vigor |
| GF34.3 | trailing, wider than tall | 30 | 46 | mid vigor |
| GF34.4 | mounded, wider than tall | 34 | 53 | mid vigor |
| GF34.5 | trailing, wider than tall | 36 | 56 | mid vigor |
| GF35.1 | upright, little spread | 35 | 35 | more upright, |
| | | | | normal |
| GF35.2 | mounded, wider than tall | 23 | 34 | more upright, |
| | | | | normal |
| GF35.3 | trailing, wider than tall | 32 | 48 | mid vigor |
| GF36 | trailing, wider than tall | 46 | 79 | monster trail, |
| | | | | high vigor |

TABLE 3
Pentas “Graffiti ® Falls” stem characteristics

| | Stem | | Stem | Stem |
|---|---|---|---|---|
| Parental | Length | Stem | Color | Internode |
| Line | (cm) | Anthocyanin | (RHS) | length (cm) |
| GF1 | 18 | Absent | 143C | 1.5 |
| GF2.1 | 10 | Absent | 143C | 0.5 |
| GF7 | 13 | Absent | 143C | 1 |
| GF11.1 | 18 | Absent | 143C | 1 |
| GF11.2 | 9 | Absent | 143C | 1 |
| GF18 | 17 | Absent | 143A | 1 |
| GF21 | 21 | Absent | 143A | 1 |
| GF22.1 | 29 | Absent | 144A | 1.5 |
| GF22.2 | 12 | Present | 143A | 1.5 |
| GF28 | 15 | Absent | 143B | 1.5 |
| GF33.1 | 14 | Absent | 143B | 1.5 |
| GF33.2 | 28 | Absent | 143B | 1.5 |
| GF34.1 | 18 | Absent | 143A | 3 |
| GF34.2 | 15 | Present | 143A | 2 |
| GF34.3 | 16 | Absent | 143B | 1.5 |
| GF34.4 | 10 | Absent | 143A | 1 |
| GF34.5 | 14 | Absent | 143A | 1 |
| GF35.1 | 9 | Absent | 143A | 0.4 |
| GF35.2 | 11 | Absent | 143A | 1 |
| GF35.3 | 12 | Absent | 143A | 1 |
| GF36 | 12 | Present | 143A | 1 |

TABLE 4
Pentas “Graffiti ® Falls” flower characteristics

| | | Petal | Petal | Corolla |
|---|---|---|---|---|
| Parental | Flower Corolla Tube Color | Length | Width | Diameter |
| Line | (RHS values) | (cm) | (cm) | (cm) |
| GF1 | 49D (Light red pink) | 0.9 | 0.5 | 2 |
| GF2.1 | 49D (Light red pink) | 0.9 | 0.5 | 2.1 |
| GF7 | N57C (Dark pink) | 0.7 | 0.4 | 1.7 |
| GF11.1 | N74B (Purple pink) | 1 | 0.6 | 2.3 |
| GF11.2 | 71A (Purple) | 1 | 0.6 | 2.4 |
| GF18 | 77A (Dark pink violet) | 0.8 | 0.5 | 2 |
| GF21 | 53C (Dark pink red) | 0.9 | 0.5 | 2 |
| GF22.1 | 52B (Red pink) | 0.7 | 0.5 | 1.8 |
| GF22.2 | 50B (Dark pink red) | 0.7 | 0.5 | 2.1 |
| GF28 | 61C (Dark pink) | 0.9 | 0.5 | 2.2 |
| GF33.1 | 53C (Dark pink red) | 1 | 0.5 | 2.4 |
| GF33.2 | 53C (Dark pink red) | 0.9 | 0.5 | 2.3 |
| GF34.1 | N79C (Purple) | 0.8 | 0.5 | 2 |
| GF34.2 | 58A (Purple) | 0.8 | 0.5 | 2.1 |
| GF34.3 | 71A (Purple) | 0.8 | 0.5 | 2 |
| GF34.4 | N79C (Purple) | 0.9 | 0.5 | 2.2 |
| GF34.5 | 61C (Dark pink) | 0.8 | 0.4 | 1.9 |
| GF35.1 | 58A (Purple) | 0.7 | 0.4 | 1.6 |
| GF35.2 | 71A (Purple) | 0.6 | 0.4 | 1.9 |
| GF35.3 | 54D (Light pink) | 0.7 | 0.4 | 1.7 |
| GF36 | 77B (Dark pink violet) | 0.7 | 0.5 | 1.9 |

### Comparisons to Most Similar Varieties

Table 5, below, compares characteristics of Pentas “Graffiti® Falls” parent lines with the characteristics of common parent Pentas clone 99/280-6 (U.S. Pat. No. 7,880,073). Column 1 lists the characteristics, column 2 shows the characteristics for Pentas “Graffiti® Falls” parent lines, and column 3 shows the characteristics for Pentas clone 99/280-6. The comparison of Pentas “Graffiti® Falls” parent lines with Pentas clone 99/280-6 was conducted in greenhouses. Briefly, Pentas “Graffiti® Falls” parent lines have an gravitropic growth habit when compared to Pentas clone 99/280-6. In addition, Pentas “Graffiti® Falls” parent lines have a variety of flower colors when compared to Pentas clone 99/280-6.

TABLE 5
Comparison of Pentas “Graffiti ® Falls”
parent lines with Pentas clone 99/280-6

| | Pentas “Graffiti ® | Pentas |
|---|---|---|
| Characteristic | Falls” parent lines | clone 99/280-6 |
| Plant phenotype/class | Trailing or basket (semi- | Prostrate; the |
| | trailing) | habit is wider |
| | | than tall, branches |
| | | are slightly arching, |
| | | limited first |
| | | order branching |
| Plant height | 18 cm to 60 cm | 25 cm |
| Plant spread | 34 cm to 79 cm | 45 cm |
| Leaf shape | Lanceolate, pointed | Lanceolate, pointed |
| Stem anthocyanin | Absent or present | Absent |
| color | | |
| Leaf size Width | 3 cm to 6 cm, up to 8 cm | Up to 3 cm |
| Flower cyme depth | 1.8 cm to 4 cm | 3.5 cm |
| Flower corolla | 1.6 cm to 2.5 cm | 1.5 cm |
| diameter | | |
| Flower corolla tube | 3 mm to 5 mm | 1 to 3 mm |
| diameter | | |
| Flower corolla tube | RHS 49D (Light red pink), | Slightly pinkish |
| color | RHS 50B (Dark pink red), | white |
| | RHS 52B (Red pink), RHS | |
| | 53C (Dark pink red), RHS | |
| | 54D (Light pink), RHS | |
| | N57C (Dark pink), RHS | |
| | 58A (Purple), RHS 61C | |
| | (Dark pink), RHS 71A | |
| | (Purple), RHS N74B | |
| | (Purple pink), RHS 77A | |
| | (Dark pink violet), | |
| | RHS 77B (Dark | |
| | pink violet), or RHS | |
| | N79C (Purple) | |

### Overview of the Pentas “Northern Lights®” Parent Lines

Pentas “Northern Lights®” parent lines differ from currently available Pentas varieties by having a range of flower colors and a range of plant heights. Pentas “Northern Lights®” parent lines are the result of numerous generations of plant selections chosen for their flower colors, plant heights, and unique inflorescence.

Pentas “Northern Lights®” parent lines were developed through a selection process that involved growing P. lanceolata as well as existing hybrid lines as comparisons to ensure continued selection toward the desired type of plant for use as a parent line. Pentas “Northern Lights®” parent lines have shown uniformity and stability, as described in the following “Objective Description of Pentas “Northern Lights®” Parent Lines” section. They have been self-pollinated a sufficient number of generations with careful attention to uniformity of plant type. The lines have been increased with continued attention to confirm transference, heritability, and stable performance as hybrid parents.

There are twenty-one Pentas “Northern Lights®” parent lines that share the common parent Pentas clone ‘99/280-6’ (U.S. Pat. No. 7,880,073), a variety having increased cold tolerance (e.g., as low as 4.0° C.), increased flowering, and unique inflorescence. Table 6 provides the Pentas “Northern Lights®” parent line designations and pedigrees. Column 1 provides the general identifier used for the parent line pedigree, which corresponds to the “Parent line designation” column in Table 1 of U.S. Provisional Application No. 63/775,505. If multiple lines have the same pedigree, this is indicated by an additional number after a period; for example NL3.1, NL3.2, and NL3.3 indicate three lines with the shared pedigree (99/280-6×(Attmarinmar/Albert1×(Felix×99/280-6/P. speciosa))). Column 2 provides the parent line designation for each Pentas “Northern Lights®” parent line. Column 3 provides the pedigree for each parent line. As noted above, Pentas clone ‘99/280-6’ (U.S. Pat. No. 7,880,073) is a shared parent in all pedigrees, and is also known as “99/280-6” or “Att”. The line “Attmarinmar” corresponds to ((99/280-6×Mauritius) (Indira×Mauritius)), which was presented in U.S. Provisional Application No. 63/775,505 as ((99/280-6×mar) (in×mar)). The line “Biattmarbi” corresponds to Bianca×99/280-6×Mauritius×Bianca, which was presented in U.S. Provisional Application No. 63/775,505 as Bi99/280-6marbi. The cross Uganda×99/280-6 may also be referred to as “Northern Lights”, but has been retained in the pedigrees for clarity.

TABLE 6
Pentas “Northern Lights ®” parent lines and pedigrees.

| General | Exemplary | Parent line | |
|---|---|---|---|
| identifier | Plant | designation | Parent line pedigree |
| NL1 | PN25-1-1562- | PN25-1- | (99/280-6/Uganda V × P. speciosa) |
| | 1959 | 1965 | |
| NL3.1 | PN25-1-1572- | PN25-1- | (99/280-6 × (Attmarinmar/Albert1 × (Felix × |
| | 1965 | 1965 | 99/280-6/P. speciosa) |
| NL3.2 | PN25-1-1573- | PN25-1- | (99/280-6 × (Attmarinmar¹/ |
| | 1965 | 1965 | Albert1 × (Felix × 99/280-6/P. speciosa |
| NL3.3 | PN25-1-1574- | PN25-1- | (99/280-6 × (Attmarinmar/Albert1 × (Felix × |
| | 1965 | 1968 | 99/280-6/P. speciosa |
| NL4 | PN25-1-1576- | PN25-1- | (Felix/99/280-6 × (Vanessa × Attmarinmar)) |
| | 1968 | 1973 | |
| NL5.1 | PN25-1-1584- | PN25-1- | (Dagmar × 99/280-6/Uganda V) |
| | 1973 | 1973 | |
| NL5.2 | PN25-1-1585- | PN25-1- | (Dagmar × 99/280-6/Uganda V) |
| | 1973 | 1974 | |
| NL6 | PN25-1-1586- | PN25-1- | (Felix × 99/280-6) |
| | 1974 | 1990 | |
| NL9 | PN25-1-1633- | PN25-1- | (Felix/99/280-6 × (Attmarinmar/Albert1 × |
| | 2016 | 2019 | (Felix × 99/280-6/P. speciosa |
| NL10 | PN25-1-1689- | PN25-1- | (99/280-6 × (Dietmar × 99/280-6/Uganda V)) |
| | 2064 | 2107 | |
| NL11 | PN25-1-1596- | PN25-1- | ((Felix/99/280-6 × (Attmarinmar/Albert1 × |
| | 1990 | 1992 | (Felix × 99/280-6/P. speciosa)) × |
| | | | (Attmarinmar/Albert1 × (Felix × 99/280- |
| | | | 6/P. speciosa) |
| NL12 | PN25-1-1603- | PN25-1- | (Attmarinmar/Balloon × Uganda × 99/280-6) |
| | 1992 | 1997 | |
| NL12 | PN25-1-1661- | PN25-1- | (Attmarinmar/Balloon × Uganda × 99/280-6) |
| | 2051 | 2056 | |
| NL13 | PN25-1-1608- | PN25-1- | (Felix/99/280-6 × (Attmarinmar/Balloon × |
| | 1997 | 2016 | Uganda × 99/280-6)) |
| NL15 | PN25-1-1738- | PN25-1- | (Dietmar × 99/280-6/Uganda V) |
| | 2119 | 2119 | |
| NL16 | PN25-1-1635- | PN25-1- | (99/280-6 × Felix/Attmarinmar) |
| | 2019 | 2031 | |
| NL18.1 | PN25-1-1641- | PN25-1- | (Attmarinmar/Albert1 × (Felix × 99/280- |
| | 2031 | 2051 | 6/P. speciosa) |
| NL18.2 | PN25-1-1672- | PN25-1- | (Attmarinmar/Albert1 × (Felix × 99/280- |
| | 2056 | 2064 | 6/P. speciosa)) |
| NL25 | PN2025-1- | PN2025-1- | Biattmarbi² × 99/280-6/Uganda V) |
| | 1725-2107 | 1959 | |
| NL26.1 | PN25-1-1741- | PN25-1- | ((Attmarinmar/Albert1) × (Felix × 99/280- |
| | 2120 | 2120 | 6/P. speciosa)) |
| NL26.2 | PN25-1-1745- | PN25-1- | ((Attmarinmar/Albert1) × (Felix × 99/280- |
| | 2122 | 2122 | 6/P. speciosa)) |

¹Attmarinmar is (((99/280-6 × Mauritius) (Indira × Mauritius)),
²Biattmarbi is Bianca × 99/280-6 × Mauritius × Bianca

### Objective Description of Pentas “Northern Lights®” Parent Lines

Pentas “Northern Lights®” parent lines have the following morphologic and other characteristics. These characteristics are based primarily on data collected in greenhouses with typical daytime temperatures of 85 Fahrenheit and typical nighttime temperatures of 75 Fahrenheit. Color references are primarily to The RHS Colour Chart of The Royal Horticultural Society of London (RHS) (5th edition). The below characteristics are characteristics shared by all Pentas “Northern Lights®” parent lines. Where characteristics vary depending on the line, this is indicated as “line-specific”. For “line-specific” characteristics, see the “Objective Description of Pentas “Northern Lights®” Parent Lines Line-specific Characteristics” section below.

### Classification:

Family: Rubiaceae
Botanical name: Pentas sp.
Common name: Pentas

### Plant:

Life cycle: Used as an annual horticulturally
Habit: Prostrate; the habit is more wider than high, branches are slightly arching,
limited first order branching
Height: 24 to 68 cm; line specific
Width: 24 to 69 cm; line specific

### Stems:

Anthocyanin color: Absent or present (dark purple to nearly black or black); line-specific
Stem length: 5 to 35 cm; line specific
Stem internode length 0.5 to 2 cm

### Leaves:

Arrangement: Pair-wise, opposite, subsequent pairs cross-wise in position
Shape: Lanceolate, pointed
Apex: Pointed
Base: Tapering
Margin: Entire
Size:
   Length: about 4.5 cm to 12 cm; line specific
   Width: 2 to 6 cm; line specific
Leaf color upper surface: RHS 137A (Moderate olive green), RHS 141B (Deep yellowish green), and RHS 139A (Dark yellowish); line specific Leaf color lower surface: RHS 137A (Moderate olive green), RHS 138A (Moderate yellowish green), and RHS 138B (Moderate yellow green)

### Flowers:

Inflorescence:
   Type: Cyme
   Diameter: In the bud stage, about 1 to 3 cm and about 5 to 9 cm when flowering.
Number of flowers: A cyme mostly composed of 10 to 60 buds and flowers, with an average of 1 to 12 flowers open at the same time; line specific
Flower opening: Sequential
Fragrance: None
Corolla:
   Shape: 5 tubular petals, long tube ending in a 5 pointed star, the pale pistil is a bit longer than the flower, anthers are positioned visibly in the tube just below the upper end and at this point the tube is a little bit wider
   Diameter: About 1.4 to 2.4 cm
   Depth: About 2.2 cm to 4.2 cm
   Tube diameter: 1 mm to 4 mm
   Tube color: RHS 56A (Light pink), RHS N57D (Dark pink), RHS 58A (Purple), RHS 61D (Dark pink), RHS 63B (Pink), RHS 64D (Pink), RHS 65B (Light pink), RHS 70B (Pink purple), RHS 72B (Pink purple), RHS N74C (Pink purple), RHS 75C (Pink violet), RHS 75D (Pink violet), RHS 76B (Light violet), RHS 77A (Dark pink violet), RHS N79C (Purple), or RHS 155C (White); line-specific
Petals:
   Texture: Smooth, glossy, lower side with a slight keel
   Size: Crown diameter about 1.4 cm to 2.4 cm; tubular at the base
   Length: About 0.5 to 1 cm
   Width: About 0.3 to 0.6 com
   Shape: Pointed, tapering
   Apex: Pointed
   Margin: Entire
Calyx (sepals):
   Shape: 5 very irregular sepals with variable length, 2 to 3 are much bigger, the others are very small and shriveled; lanceolate, quite similar to leaves.
   Apex: Pointed
   Margin: Entire
   Color of upper surface: RHS 137A (Moderate olive green) and RHS 138A (Moderate yellowish green)
   Color of lower surface: RHS 138B (Moderate yellow green) and RHS 137C (Moderate yellow green)
Pedicel:
   Length: About 1 mm to 2 mm
   Diameter: About 1 mm

### Objective Description of Pentas “Northern Lights®” Parent Lines Line-Specific Characteristics

The below characteristics are line-specific to particular Pentas “Northern Lights®” parent lines. Each characteristic is uniform and stable within the line where it is present. In other words, each Pentas “Northern Lights®” parent line is true to type for its specific characteristic. Table 7 provides line-specific plant characteristics, Table 8 provides line-specific stem characteristics, and Table 9 provides line-specific flower characteristics.

TABLE 7
Pentas “Northern Lights ®” line-specific plant characteristics

| | | Plant | Plant | |
|---|---|---|---|---|
| Parental | | Height | width | |
| Line | Plant Habit | (cm) | (cm) | Plant Spread |
| NL1 | mounded, taller than wide | 42 | 43 | Northern Lights, tall |
| NL3.1 | mounded, taller than wide | 39 | 39 | Northern Lights, tall |
| NL3.2 | upright, little spread | 61 | 46 | Northern Lights, tall |
| NL3.3 | mounded, taller than wide | 44 | 41 | Northern Lights, tall |
| NL4 | upright, little spread | 44 | 44 | Northern Lights, tall |
| NL5.1 | mounded, taller than wide | 20 | 37 | Northern Lights, |
| | | | | dwarf |
| NL5.2 | mounded, taller than wide | 21 | 24 | Northern Lights, |
| | | | | dwarf |
| NL6 | upright, little spread | 66 | 45 | Northern Lights, tall |
| NL9 | upright, little spread | 55 | 35 | Northern Lights, tall |
| NL10 | mounded, taller than wide | 38 | 47 | Northern Lights, |
| | | | | more upright |
| NL11 | upright, little spread | 68 | 56 | Northern Lights, tall |
| NL12 | upright, little spread | 66 | 69 | Northern Lights, tall |
| NL12 | mounded, taller than wide | 35 | 52 | Northern Lights, tall |
| NL13 | upright, little spread | 67 | 45 | Northern Lights, tall |
| NL15 | upright, little spread | 25 | 38 | Northern Lights, |
| | | | | dwarf |
| NL16 | mounded, taller than wide | 53 | 45 | Northern Lights, tall |
| NL18.1 | mounded, wider than tall | 15 | 28 | Northern Lights, |
| | | | | dwarf |
| NL18.2 | upright, little spread | 31 | 35 | Northern Lights, |
| | | | | dwarf |
| NL25 | upright, little spread | 42 | 33 | Northern Lights, tall |
| NL26.1 | mounded, wider than tall | 37 | 48 | Northern Lights, mid |
| NL26.2 | mounded, taller than wide | 38 | 49 | Northern Lights, mid |

TABLE 8
Pentas “Northern Lights ®” line-specific stem characteristics

| Parental | Stem Length | Stem | Stem Color | Stem Internode |
|---|---|---|---|---|
| Line | (cm) | Anthocyanin | (RHS) | length (cm) |
| NL1 | 15 | Present | 138A | 0.5 |
| NL3.1 | 12 | Absent | 138A | 1 |
| NL3.2 | 18 | Absent | 138B | 2.5 |
| NL3.3 | 17 | Absent | 145 | 1 |
| NL4 | 16 | Present | 138B | 0.8 |
| NL5.1 | 11 | Absent | 138B | 1 |
| NL5.2 | 13 | Absent | 138B | 1 |
| NL6 | 22 | Absent | 138A | 1.5 |
| NL9 | 15 | Absent | 138B | 1.5 |
| NL10 | 16 | Present | 138A | 1 |
| NL11 | 31 | Absent | 138B | 2 |
| NL12 | 33 | Absent | 143A | 0.5 |
| NL12 | 6 | Absent | 143A | 1 |
| NL13 | 35 | Absent | 143 | 1 |
| NL15 | 8 | Absent | 138A | 0.5 |
| NL16 | 32 | Absent | 144 | 1.5 |
| NL18.1 | 5 | Absent | 143A | 0.5 |
| NL18.2 | 13 | Absent | 143A | 1 |
| NL25 | 11 | Present | 144A | 1.5 |
| NL26.1 | 13 | Absent | 143B | 1.5 |
| NL26.2 | 16 | Absent | 143C | 1 |

TABLE 9
Pentas “Northern Lights ®” line-specific flower characteristics

| | Flower Corolla Tube | | | | |
|---|---|---|---|---|---|
| | Color (RHS | Petal | Petal | Corolla | Corolla |
| Parental | value)/Flower Color | Length | Width | Diameter | Depth |
| Line | Description | (cm) | (cm) | (cm) | (cm) |
| NL1 | 155D (White) | 0.6 | 0.4 | 1.5 | 4 |
| NL3.1 | 155C (White) | 0.6 | 0.5 | 1.6 | 4.1 |
| NL3.2 | 56A (Light pink) | 0.8 | 0.5 | 1.9 | 4.1 |
| NL3.3 | 76B (Light violet) | 0.8 | 0.6 | 2 | 3.8 |
| NL4 | 77A (Dark pink violet) | 0.6 | 0.4 | 1.4 | 2.9 |
| NL5.1 | 75C (Pink violet) | 0.6 | 0.4 | 1.6 | 4.2 |
| NL5.2 | Pink color | N/A | N/A | N/A | N/A |
| NL6 | 75D (Pink violet) | 0.6 | 0.5 | 1.5 | 3.7 |
| NL9 | 65B (Light pink) | 0.7 | 0.4 | 1.9 | 3.8 |
| NL10 | Pure white, no | N/A | N/A | N/A | N/A |
| | noticeable blush | | | | |
| NL11 | 72B (Pink purple) | 0.5 | 0.5 | 1.4 | 3.5 |
| NL12 | 70B (Pink purple) | 0.8 | 0.4 | 1.8 | 3.5 |
| NL12 | N57D (Dark pink) | 0.7 | 0.4 | 1.8 | 3.4 |
| NL13 | N74C (Pink purple) | 1 | 0.5 | 2.4 | 4.2 |
| NL15 | Red flower color with | N/A | N/A | N/A | N/A |
| | red stigmas | | | | |
| NL16 | 61D (Dark pink) | 1 | 0.6 | 2.4 | 3.9 |
| NL18.1 | 58A (Purple) | 0.6 | 0.3 | 1.4 | 2.5 |
| NL18.2 | N79C (Purple) | 0.8 | 0.5 | 2 | 3.8 |
| NL25 | 77A (Dark pink violet) | 0.5 | 0.3 | 1.4 | 2.2 |
| NL26.1 | 64D (Pink) | 0.7 | 0.6 | 1.9 | 3.9 |
| NL26.2 | 63B (Pink) | 0.8 | 0.6 | 2 | 3.6 |

### Comparisons to Most Similar Varieties

Table 10, below, compares characteristics of Pentas “Northern Lights®” parent lines with the characteristics of common parent Pentas clone 99/280-6 (U.S. Pat. No. 7,880,073). Column 1 lists the characteristics, column 2 shows the characteristics for Pentas “Northern Lights®” parent lines, and column 3 shows the characteristics for Pentas clone 99/280-6. The comparison of Pentas “Northern Lights®” parent lines with Pentas clone 99/280-6 was conducted in greenhouses. Briefly, Pentas “Northern Lights®” parent lines have a wide variety of flower colors when compared to Pentas clone 99/280-6. In addition, Pentas “Northern Lights®” parent lines have varied plant height and spread when compared to Pentas clone 99/280-6.

TABLE 10
Comparison of Pentas “Northern Lights ®”
parent lines with Pentas clone 99/280-6

| | Pentas “Northern | Pentas |
|---|---|---|
| Characteristic | Lights ®” parent lines | clone 99/280-6 |
| Plant height | 24 cm to 68 cm | 25 cm |
| Plant spread | 24 cm to 69 cm | 45 cm |
| Leaf shape | Lanceolate, pointed | Lanceolate, pointed |
| Stem anthocyanin | Absent or present | Absent |
| color | | |
| Leaf size Width | 2 cm to 6 cm | Up to 3 cm |
| Flower cyme depth | 2.5 cm to 5 cm | 3.5 cm |
| Flower corolla | 1.4 cm to 2.4 cm | 1.5 cm |
| diameter | | |
| Flower corolla tube | 2 mm to 4 mm | 1 mm to 3 mm |
| diameter | | |
| Corolla tube color | RHS 56A (Light pink), RHS | Slightly pinkish |
| | N57D (Dark pink), RHS 58A | white |
| | (Purple), RHS 61D (Dark | |
| | pink), RHS 63B (Pink), RHS | |
| | 64D (Pink), RHS 65B (Light | |
| | pink), RHS 70B (Pink | |
| | purple), RHS 72B (Pink | |
| | purple), RHS N74C (Pink | |
| | purple), RHS 75C (Pink | |
| | violet), RHS 75D (Pink | |
| | violet), RHS 76B (Light | |
| | violet), RHS 77A (Dark pink | |
| | violet), RHS N79C (Purple), | |
| | or RHS 155C (White) | |

### Further Embodiments

Pentas is an important and valuable flowering plant. Thus, a continuing goal of Pentas plant breeders is to develop stable inbred Pentas parent lines with attractive characteristics that are horticulturally sound for use in producing hybrid Pentas. To accomplish this goal, the Pentas breeder must select and develop Pentas plants with traits that result in superior parent lines. Further, the Pentas breeder must identify traits not yet present in the market that will be valuable to customers.

The present disclosure provides Pentas “Graffiti® Falls” parent lines and Pentas “Northern Lights®” parent lines. The Pentas “Graffiti® Falls” parent lines provide an attractive gravitropic growth habit which may be suitable, for example, in pot and hanging basket growth. The Pentas “Northern Lights®” parent lines provide a range of vibrant new flower colors not previously disclosed. Both of these new parent line groups are suitable for use in producing new Pentas hybrid cultivars with the commercially valuable traits of gravitropic growth habits and new flower colors.

### Plant Breeding and Selection

Proper testing should detect any major faults and establish the level of superiority or improvement over current varieties. In addition to showing superior performance, there must be a demand for a new variety that is compatible with industry standards or which creates a new market. The introduction of a new variety will incur additional costs to the seed producer, the grower, processor and consumer for special advertising and marketing, altered seed and commercial production practices, and new product utilization. The testing preceding release of a new variety should take into consideration research and development costs as well as technical superiority of the final variety. For seed-propagated varieties, it must be feasible to produce seed easily and economically.

There are numerous steps in the development of any novel, desirable plant germplasm. Plant breeding begins with the analysis and definition of problems and weaknesses of the current germplasm, the establishment of program goals, and the definition of specific breeding objectives. The next step is selection of germplasm that possess the traits to meet the program goals. The goal is to combine in a single variety an improved combination of desirable traits from the parental germplasm. These important traits may include increased number of flowers, resistance to diseases and insects, better stems and roots, tolerance to drought and heat, and better horticultural quality.

Choice of breeding or selection methods depends on the mode of plant reproduction, the heritability of the trait(s) being improved, and the type of cultivar used commercially (e.g., F₁ hybrid variety, pureline variety, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

The complexity of inheritance influences choice of the breeding method. Backcross breeding is used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program should include a periodic, objective evaluation of the efficiency of the breeding procedure. Evaluation criteria vary depending on the goal and objectives, but should include gain from selection per year based on comparisons to an appropriate standard, the overall value of the advanced breeding lines, and the number of successful cultivars produced per unit of input (e.g., per year, per dollar expended, etc.).

Promising advanced breeding lines are thoroughly tested and compared to appropriate standards in environments representative of the commercial target area(s) for at least three years. The best lines are candidates for new commercial cultivars. Those still deficient in a few traits are used as parents to produce new populations for further selection.

A most difficult task is the identification of individuals that are genetically superior, because for most traits the true genotypic value is masked by other confounding plant traits or environmental factors. One method of identifying a superior plant is to observe its performance relative to other experimental plants and to a widely grown standard cultivar. If a single observation is inconclusive, replicated observations provide a better estimate of its genetic worth.

The goal of Pentas plant breeding is to develop new, unique and superior Pentas varieties and hybrids. The breeder initially selects and crosses two or more parental lines, followed by repeated selfing and selection, producing many new genetic combinations. The breeder can theoretically generate billions of different genetic combinations via crossing, selfing and mutations. The breeder has no direct control at the cellular level. Therefore, two breeders will never develop the same line, or even very similar lines, having the same Pentas traits.

Each growing season, the plant breeder selects the germplasm to advance to the next generation. This germplasm is grown under different geographical, climatic, and soil conditions, and further selections are then made during, and at the end of, the growing season. The varieties that are developed are unpredictable. This unpredictability is because the breeder's selection occurs in unique environments, with no control at the DNA level (using conventional breeding procedures), and with millions of different possible genetic combinations being generated. A breeder of ordinary skill in the art cannot predict the final resulting lines he develops, except possibly in a very gross and general fashion. The same breeder cannot produce the same line twice by using the exact same original parents and the same selection techniques. This unpredictability results in the expenditure of large research monies to develop superior new plant varieties.

Pedigree breeding and recurrent selection breeding methods are used to develop varieties from breeding populations. Breeding programs combine desirable traits from two or more varieties or various broad-based sources into breeding pools from which varieties are developed by selfing and selection of desired phenotypes. The new varieties are evaluated to determine which have commercial potential.

Pedigree breeding is used commonly for the improvement of self-pollinating crops or inbred lines of cross-pollinating crops. Two parents which possess favorable, complementary traits are crossed to produce an F₁. An F₂ population is produced by selfing one or several F₁s or by intercrossing two F₁s (sib mating). Selection of the best individuals is usually begun in the F₂ population. Then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new varieties.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding has been used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines with each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques available for the analysis, comparison and characterization of plant genotype. Such techniques include, without limitation, DNA- or RNA-sequencing, CAPS Markers, ELISA, Western blot, microarrays, Single Nucleotide Polymorphisms (SNPs), Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), Differential Display Polymerase Chain Reaction (DD-PCR), Quantitative Real-Time Polymerase Chain Reaction (qRT-PCR), DNA Amplification Fingerprinting (DAF), Sequence Characterized Amplified Regions (SCARs), Amplified Fragment Length Polymorphisms (AFLPs), and Simple Sequence Repeats (SSRs, which are also referred to as Microsatellites).

Molecular markers, which include markers identified through the use of techniques such as Isozyme Electrophoresis, RFLPs, RAPDs, AP-PCR, DAF, SCARs, AFLPs, SSRs, and SNPs, may be used in plant breeding. One use of molecular markers is Quantitative Trait Loci (QTL) mapping. QTL mapping is the use of markers which are known to be closely linked to alleles that have measurable effects on a quantitative trait. Selection in the breeding process is based upon the accumulation of markers linked to the positive effecting alleles and/or the elimination of the markers linked to the negative effecting alleles from the plant's genome.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Molecular markers may also be used to identify and exclude certain sources of germplasm as parental varieties or ancestors of a plant by providing a means of tracking genetic profiles through crosses.

Mutation breeding is another method of introducing new traits into Pentas varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid, or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company (1993).

The production of double haploids can also be used for the development of homozygous varieties in a breeding program. Double haploids are produced by the doubling of a set of chromosomes from a heterozygous plant to produce a completely homozygous individual. For example, see Wan, et al., Theor. Appl. Genet., 77:889-892 (1989).

Descriptions of other breeding methods that are commonly used for different traits and crops can be found in one of several reference books (e.g., Principles of Plant Breeding, John Wiley and Son, pp. 115-161 (1960); Allard (1960); Simmonds (1979); Sneep, et al. (1979); Fehr (1987); “Carrots and Related Vegetable Umbelliferae,” Rubatzky, V. E., et al. (1999)).

### Molecular Biological Techniques

With the advent of molecular biological techniques that have allowed the isolation and characterization of genes that encode specific protein products, scientists in the field of plant biology developed a strong interest in engineering the genome of plants to contain and express foreign genes, or additional, or modified versions of native, or endogenous, genes (perhaps driven by different promoters) in order to alter the traits of a plant in a specific manner. Any DNA sequences, whether from a different species or from the same species, which are introduced into the genome using transformation or various breeding methods, are referred to herein collectively as “transgenes.” Over the last fifteen to twenty years, several methods for producing transgenic plants have been developed, and the present invention, in particular embodiments, also relates to transformed versions of the claimed lines.

Nucleic acids or polynucleotides refer to RNA or DNA that is linear or branched, single or double stranded, or a hybrid thereof. The term also encompasses RNA/DNA hybrids. These terms also encompass untranslated sequence located at both the 3′ and 5′ ends of the coding region of the gene: at least about 1000 nucleotides of sequence upstream from the 5′ end of the coding region and at least about 200 nucleotides of sequence downstream from the 3′ end of the coding region of the gene. Less common bases, such as inosine, 5-methylcytosine, 6-methyladenine, hypoxanthine, and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The antisense polynucleotides and ribozymes can consist entirely of ribonucleotides, or can contain mixed ribonucleotides and deoxyribonucleotides. The polynucleotides of the invention may be produced by any means, including genomic preparations, cDNA preparations, in vitro synthesis, RT-PCR, and in vitro or in vivo transcription.

Plant transformation involves the construction of an expression vector that will function in plant cells. Such a vector comprises DNA comprising a gene under control of, or operatively linked to, a regulatory element (for example, a promoter). The expression vector may contain one or more such operably linked gene/regulatory element combinations. The vector(s) may be in the form of a plasmid, and can be used alone or in combination with other plasmids, to provide transformed Pentas plants using transformation methods as described below to incorporate transgenes into the genetic material of the Pentas plant(s).

### Gene Conversion and Further Breeding Methods

When the term “Pentas plant” is used in the context of the present disclosure, this also includes any gene conversions of that variety. The term “gene converted plant” as used herein refers to those Pentas plants which are developed by backcrossing, genetic engineering, or mutation, wherein essentially all of the desired morphological and physiological characteristics of a variety are recovered in addition to the one or more genes transferred into the variety via the backcrossing technique, genetic engineering, or mutation. Backcrossing methods can be used with the present invention to improve or introduce a characteristic into the variety. The term “backcrossing” as used herein refers to the repeated crossing of a hybrid progeny back to the recurrent parent, i.e., backcrossing 1, 2, 3, 4, 5, 6, 7, 8, 9, or more times to the recurrent parent. The parental Pentas plant which contributes the gene for the desired characteristic is termed the “nonrecurrent” or “donor parent.” This terminology refers to the fact that the nonrecurrent parent is used one time in the backcross protocol and therefore does not recur. The parental Pentas plant to which the gene or genes from the nonrecurrent parent are transferred is known as the recurrent parent as it is used for several rounds in the backcrossing protocol. Poehlman & Sleper (1994) and Fehr (1993). In a typical backcross protocol, the original variety of interest (recurrent parent) is crossed to a second variety (nonrecurrent parent) that carries the gene of interest to be transferred. The resulting progeny from this cross are then crossed again to the recurrent parent and the process is repeated until a Pentas plant is obtained wherein essentially all of the desired morphological and physiological characteristics of the recurrent parent are recovered in the converted plant, in addition to the transferred gene from the nonrecurrent parent.

The selection of a suitable recurrent parent is an important step for a successful backcrossing procedure. The goal of a backcross protocol is to alter or substitute a trait or characteristic in the original line. To accomplish this, a gene of the recurrent cultivar is modified or substituted with the desired gene from the nonrecurrent parent, while retaining essentially all of the rest of the desired genetic, and therefore the desired physiological and morphological characteristics of the original line. The choice of the particular nonrecurrent parent will depend on the purpose of the backcross. One of the major purposes is to add some commercially desirable, agronomically important trait to the plant. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

New varieties can also be developed from more than two parents. The technique, known as modified backcrossing, uses different recurrent parents during the backcrossing. Modified backcrossing may be used to replace the original recurrent parent with a variety having certain more desirable characteristics or multiple parents may be used to obtain different desirable characteristics from each.

In addition to being used to create a backcross conversion, backcrossing can also be used in combination with pedigree breeding. As discussed previously, backcrossing can be used to transfer one or more specifically desirable traits from one variety, the donor parent, to a developed variety called the recurrent parent, which has overall good agronomic characteristics yet lacks that desirable trait or traits. However, the same procedure can be used to move the progeny toward the genotype of the recurrent parent, but at the same time retain many components of the nonrecurrent parent by stopping the backcrossing at an early stage and proceeding with selfing and selection. For example, an inbred Pentas line may be crossed with another variety to produce a first generation progeny plant. The first generation progeny plant may then be backcrossed to one of its parent varieties to create a BC1 or BC2. Progeny are selfed and selected so that the newly developed variety has many of the attributes of the recurrent parent and yet several of the desired attributes of the nonrecurrent parent. This approach leverages the value and strengths of the recurrent parent for use in new Pentas varieties.

Many gene traits have been identified that are not regularly selected in the development of a new line but that can be improved by backcrossing techniques. Gene traits may or may not be transgenic. Examples of these traits include, but are not limited to, male sterility, modified fatty acid metabolism, modified carbohydrate metabolism, herbicide resistance or tolerance, resistance or tolerance for bacterial, fungal, or viral disease, insect resistance or tolerance, stress tolerance and/or resistance, industrial usage, yield stability, yield enhancement, physical appearance, drydown, standability, prolificacy, sex determination, improved agronomic quality, improved flower color, improved flower quality and/or quantity, modified phytic acid metabolism, modified protein metabolism, improved plant color, improved plant size, improved habit size and shape, improved leaf size and shape, improved seasonal performance, improved branching, improved flowering, and the like. These genes are generally inherited through the nucleus. Several of these gene traits are described in U.S. Pat. Nos. 5,777,196, 5,948,957, and 5,969,212, the disclosures of which are specifically hereby incorporated by reference.

With the advent of molecular biological techniques that have allowed the isolation and characterization of genes that encode specific protein products, scientists in the field of plant biology developed a strong interest in engineering the genome of plants to contain and express foreign genes, or additional, or modified versions of native, or endogenous, genes (perhaps driven by different promoters) in order to alter the traits of a plant in a specific manner. Such foreign additional and/or modified genes are referred to herein collectively as “transgenes.” Over the last fifteen to twenty years several methods for producing transgenic plants have been developed, and the present invention, in particular embodiments, also relates to transformed versions of the claimed hybrid.

### Tissue Culture

Further reproduction of the variety can occur by tissue culture and regeneration. For example see Caprini, G. P., et al., 2004, Initiation and metabolomic investigation of plant cell suspension cultures of Pentas lanceolata (Rubiaceae), XXVI RESEM, Instituto de Quimica, Universidade Federal Fluminense. It is clear from the literature that the state of the art is such that these methods of obtaining plants are “conventional” in the sense that they are routinely used and have a very high rate of success. Thus, another aspect of this invention is to provide cells which upon growth and differentiation produce Pentas plants having the physiological and morphological characteristics of Pentas “Northern Lights®” parent lines and Pentas “Graffiti® Falls” parent lines.

As used herein, the term “tissue culture” indicates a composition comprising isolated cells of the same or a different type or a collection of such cells organized into parts of a plant. Exemplary types of tissue cultures are protoplasts, calli, meristematic cells, and plant cells that can generate tissue culture that are intact in plants or parts of plants, such as leaves, pollen, ovules, embryos, roots, root tips, anthers, pistils, stems, flowers, seeds, petioles, fruit, cells, cotyledon, hypocotyl, meristematic cells, portions thereof, and the like. Means for preparing and maintaining plant tissue culture are well known in the art. By way of example, a tissue culture including organs has been used to produce regenerated plants. U.S. Pat. Nos. 5,959,185; 5,973,234 and 5,977,445 describe certain techniques, the disclosures of which are incorporated herein by reference.

Transformation of plant protoplasts also can be achieved using methods based on calcium phosphate precipitation, polyethylene glycol treatment, electroporation, and combinations of these treatments (see, e.g., Potrykus et al., 1985; Omirulleh et al., 1993; Fromm et al., 1986; Uchimiya et al., 1986; Marcotte et al., 1988). Transformation of plants and expression of foreign genetic elements is exemplified in Choi et al. (1994) and Ellul et al. (2003). Direct uptake of DNA into protoplasts using CaCl2 precipitation, polyvinyl alcohol, or poly-L-ornithine has also been reported (see, e.g., Hain et al., 1985 and Draper et al., 1982). Electroporation of protoplasts and whole cells and tissues have also been described (see, e.g., Saker et al., 1998; Donn et al., 1990; D'Halluin et al., 1992; and Laursen et al., 1994; Chupean et al., 1989).

As used herein, the term “plant” includes plant cells, plant protoplasts, plant cell tissue cultures from which Pentas plants can be regenerated, plant calli, plant clumps and plant cells that are intact in plants or parts of plants, such as fruit, leaves, pollen, embryos, cotyledons, hypocotyl, roots, root tips, anthers, pistils, flowers, seeds, stems, cells, portions thereof, and the like.

The use of the terms “a,” “an,” and “the,” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While a number of exemplary aspects and embodiments have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope.

### DEPOSIT INFORMATION

### Pentas Line ‘PN25-1-2141’

A deposit of at least 625 seeds of the Pentas line ‘PN25-1-2141’ was made with the American Type Culture Collection (ATCC), ATCC Patent Depository, 10801 University Boulevard, Manassas, Virginia, 20110, USA, and assigned ATCC number X6. The seeds deposited with the ATCC on DATE were obtained from the seed of the variety maintained by Ernst Benary Samenzucht GmbH, having an address at Friedrich-Benary-Weg 1, 34346 Hann. Münden, Germany since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the Pentas line ‘PN25-1-2141’ will be maintained in the ATCC, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

### Pentas Line ‘PN25-1-2513’

A deposit of at least 625 seeds of the Pentas line ‘PN25-1-2513’ was made with the American Type Culture Collection (ATCC), ATCC Patent Depository, 10801 University Boulevard, Manassas, Virginia, 20110, USA, and assigned ATCC number X7. The seeds deposited with the ATCC on DATE were obtained from the seed of the variety maintained by Ernst Benary Samenzucht GmbH, having an address at Friedrich-Benary-Weg 1, 34346 Hann. Münden, Germany since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the Pentas line ‘PN25-1-2513’ will be maintained in the ATCC, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

### Pentas Line ‘PN25-1-1882-2249’

A deposit of at least 625 seeds of the Pentas line ‘PN25-1-2249’ was made with the American Type Culture Collection (ATCC), ATCC Patent Depository, 10801 University Boulevard, Manassas, Virginia, 20110, USA, and assigned ATCC number X8. The seeds deposited with the ATCC on DATE were obtained from the seed of the variety maintained by Ernst Benary Samenzucht GmbH, having an address at Friedrich-Benary-Weg 1, 34346 Hann. Münden, Germany since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the Pentas line ‘PN25-1-2249’ will be maintained in the ATCC, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

### Pentas Line ‘PN25-1-2346’

A deposit of at least 625 seeds of the Pentas line ‘PN25-1-2346’ was made with the American Type Culture Collection (ATCC), ATCC Patent Depository, 10801 University Boulevard, Manassas, Virginia, 20110, USA, and assigned ATCC number X9. The seeds deposited with the ATCC on DATE were obtained from the seed of the variety maintained by Ernst Benary Samenzucht GmbH, having an address at Friedrich-Benary-Weg 1, 34346 Hann. Münden, Germany since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the Pentas line ‘PN25-1-2346’ will be maintained in the ATCC, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

### Pentas Line ‘PN25-1-2358’

A deposit of at least 625 seeds of the Pentas line ‘PN25-1-2358’ was made with the American Type Culture Collection (ATCC), ATCC Patent Depository, 10801 University Boulevard, Manassas, Virginia, 20110, USA, and assigned ATCC number X10. The seeds deposited with the ATCC on DATE were obtained from the seed of the variety maintained by Ernst Benary Samenzucht GmbH, having an address at Friedrich-Benary-Weg 1, 34346 Hann. Münden, Germany since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the Pentas line ‘PN25-1-2358’ will be maintained in the ATCC, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

### Pentas Line ‘PN25-1-1959’

A deposit of at least 625 seeds of the Pentas line ‘PN25-1-1959’ was made with the American Type Culture Collection (ATCC), ATCC Patent Depository, 10801 University Boulevard, Manassas, Virginia, 20110, USA, and assigned ATCC number X1. The seeds deposited with the ATCC on DATE were obtained from the seed of the variety maintained by Ernst Benary Samenzucht GmbH, having an address at Friedrich-Benary-Weg 1, 34346 Hann. Münden, Germany since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the Pentas line ‘PN25-1-1959’ will be maintained in the ATCC, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

### Pentas Line ‘PN25-1-1965’

A deposit of at least 625 seeds of the Pentas line ‘PN25-1-1965’ was made with the American Type Culture Collection (ATCC), ATCC Patent Depository, 10801 University Boulevard, Manassas, Virginia, 20110, USA, and assigned ATCC number X3. The seeds deposited with the ATCC on DATE were obtained from the seed of the variety maintained by Ernst Benary Samenzucht GmbH, having an address at Friedrich-Benary-Weg 1, 34346 Hann. Münden, Germany since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the Pentas line ‘PN25-1-1965’ will be maintained in the ATCC, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

### Pentas Line ‘PN25-1-1968’

A deposit of at least 625 seeds of the Pentas line ‘PN25-1-1968’ was made with the American Type Culture Collection (ATCC), ATCC Patent Depository, 10801 University Boulevard, Manassas, Virginia, 20110, USA, and assigned ATCC number X4. The seeds deposited with the ATCC on DATE were obtained from the seed of the variety maintained by Ernst Benary Samenzucht GmbH, having an address at Friedrich-Benary-Weg 1, 34346 Hann. Münden, Germany since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the Pentas line ‘PN25-1-1968’ will be maintained in the ATCC, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

### Pentas Line ‘PN25-1-1973’

A deposit of at least 625 seeds of the Pentas line ‘PN25-1-1973’ was made with the American Type Culture Collection (ATCC), ATCC Patent Depository, 10801 University Boulevard, Manassas, Virginia, 20110, USA, and assigned ATCC number X5. The seeds deposited with the ATCC on DATE were obtained from the seed of the variety maintained by Ernst Benary Samenzucht GmbH, having an address at Friedrich-Benary-Weg 1, 34346 Hann. Münden, Germany since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the Pentas line ‘PN25-1-1973’ will be maintained in the ATCC, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

What is claimed is:

1. A Pentas parent line plant comprising:
(a) an gravitropic growth habit;
(b) a branch shoot length of about 1 cm to about 5.5 cm;
(c) a leaf width of about 3 cm to about 8 cm;
(d) a flower corolla diameter of about 1.6 cm to about 2.4 cm; and
(e) a stem color selected from the group consisting of RHS 143A, RHS 143B, and RHS 143C.

2. The plant of claim 1, further comprising a flower corolla tube color selected from the group consisting of: RHS 49D, RHS 50B, RHS 52B, RHS 53C, RHS 54D, RHS N57C, RHS 58A, RHS 61C, RHS 71A, RHS N74B, RHS 77A, RHS 77B, and RHS N79C.

3. A plant part from the plant of claim 1, wherein said part is a leaf, a flower, an ovule, a pollen grain, a seed, a fruit, a cell, or a portion thereof.

4. The plant part of claim 3, wherein said part is a flower.

5. A Pentas plant having all the physiological and morphological characteristics of the plant of claim 1.

6. A plant part from the plant of claim 5, wherein said part is a leaf, a flower, an ovule, a pollen grain, a seed, a fruit, a cell, or a portion thereof.

7. The plant part of claim 6, wherein said part is a flower.

8. An F1 hybrid Pentas plant having the Pentas parent line plant of claim 1 as a parent.

9. The plant part of claim 6, wherein said part is a seed selected from the group consisting of:
a Pentas seed designated as ‘PN25-1-2141’, representative sample of seed having been deposited under ATCC Accession Number X6;
a Pentas seed designated as ‘PN25-1-2513’, representative sample of seed having been deposited under ATCC Accession Number X7;
a Pentas seed designated as ‘PN25-1-2249’, representative sample of seed having been deposited under ATCC Accession Number X8;
a Pentas seed designated as ‘PN25-1-2346’, representative sample of seed having been deposited under ATCC Accession Number X9; and
a Pentas seed designated as ‘PN25-1-2358’, representative sample of seed having been deposited under ATCC Accession Number X10.

10. A tissue or cell culture produced from protoplasts or cells from the plant of claim 1, wherein said cells or protoplasts are produced from a plant part selected from the group consisting of root, root tip, meristematic cell, stem, hypocotyl, petiole, cotyledon, leaf, flower, anther, pollen, pistil, embryo, seed, and fruit.

11. A Pentas plant regenerated from the tissue or cell culture of claim 10, wherein the plant has all of the morphological and physiological characteristics of a Pentas parent line plant of claim 1.

12. A method of producing a seed of a Pentas parent line-derived plant, comprising:
(a) crossing a first Pentas parent line plant with a second Pentas parent line plant, the first Pentas plant comprising:
(i) an gravitropic growth habit;
(ii) a branch shoot length of about 1 cm to about 5.5 cm;
(iii) a leaf width of about 3 cm to about 8 cm;
(iv) a flower corolla diameter of about 1.6 cm to about 2.4 cm; and
(v) a stem color selected from the group consisting of RHS 143A, RHS 143B, and RHS 143C,
whereby seed of a Pentas parent line-derived plant forms.

13. The method of claim 12, further comprising:
(b) crossing a plant grown from the Pentas parent line-derived plant seed with itself or a second Pentas parent line-derived plant to yield additional Pentas parent line-derived plant-derived seed;
(c) growing the additional Pentas parent line-derived seed of step (b) to yield additional Pentas parent line-derived plants; and
(d) repeating the crossing and growing of steps (b) and (c) for an additional 3-10 generations to generate further Pentas parent line-derived plants.

14. A method of making Pentas seeds, said method comprising crossing the plant of claim 1 with another Pentas plant and harvesting F1 seed therefrom.

15. The method of claim 14, further comprising:
(a) selecting one or more of the F1 progeny for plants that have a desired trait to produce selected progeny plants;
(b) crossing the selected progeny plants with a Pentas parent line comprising:
(i) an gravitropic growth habit;
(ii) a branch shoot length of about 1 cm to about 5.5 cm;
(iii) a leaf width of about 3 cm to about 8 cm;
(iv) a flower corolla diameter of about 1.6 cm to about 2.4 cm; and
(v) a stem color selected from the group consisting of RHS 143A, RHS 143B, and RHS 143C,
thereby producing backcross progeny plants;
(c) selecting for backcross progeny plants that have the desired trait and all of the physiological and morphological characteristics of a Pentas parent line when grown in the same environmental conditions to produce selected backcross progeny plants; and
(d) repeating steps (b) and (c) three or more times in succession to produce selected fourth or higher backcross progeny plants that comprise the desired trait and all of the physiological and morphological characteristics of a Pentas parent line when grown in the same environmental conditions.

16. The method of claim 15, wherein the desired trait is herbicide resistance, insect resistance, resistance for bacterial, fungal, or viral disease, improved horticultural quality, or color.

17. A Pentas parent line plant comprising:
(a) a flower color characteristic selected from the group consisting of RHS 56A (Light pink), RHS N57D (Dark pink), RHS 58A (Purple), RHS 61D (Dark pink), RHS 63B (Pink), RHS 64D (Pink), RHS 65B (Light pink), RHS 70B (Pink purple), RHS 72B (Pink purple), RHS N74C (Pink purple), RHS 75C (Pink violet), RHS 75D (Pink violet), RHS 76B (Light violet), RHS 77A (Dark pink violet), or RHS N79C (Purple); and
(b) a branch shoot length of about 1 cm to about 6.5 cm.

18. A plant part from the plant of claim 17, wherein said part is a leaf, a flower, an ovule, a pollen grain, a seed, a fruit, a cell, or a portion thereof.

19. A method of making Pentas seeds, said method comprising crossing the plant of claim 17 with another Pentas plant and harvesting seed therefrom.

20. The plant part of claim 18, wherein said part is a seed selected from the group consisting of:
a Pentas seed designated as ‘PN25-1-1959’, representative sample of seed having been deposited under ATCC Accession Number X1;
a Pentas seed designated as ‘PN25-1-1965’, representative sample of seed having been deposited under ATCC Accession Number X3;
a Pentas seed designated as ‘PN25-1-1968’, representative sample of seed having been deposited under ATCC Accession Number X4; and
a Pentas seed designated as ‘PN25-1-1973’, representative sample of seed having been deposited under ATCC Accession Number X5.
